(19) United States
(12) Patent Application Publication
(10) Pub. No.: US 2006/0088487 A1
(43) Pub. Date: Apr. 27, 2006

(54) Block ethylenic copolymers comprising a vinyllactam block, cosmetic or pharmaceutical compositions containing them and cosmetic use of these copolymers

(76) Inventor: Nathalie Mougin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/142,912

(22) Filed: Jun. 2, 2005

Related U.S. Application Data
(63) Continuation of application No. 10/940,756, filed on Sep. 15, 2004, now abandoned.

(30) Foreign Application Priority Data
Sep. 15, 2003 (FR) 03 50538

(51) Int. Cl. A61K 8/00

(52) U.S. Cl. 424/70.1

(57) ABSTRACT

Disclosed herein are a linear block ethylenic copolymer comprising:
at least one block A obtained from monomers comprising from 52% to 99% by weight of at least one ethylenic monomer comprising a lactam ring, of formula (I): 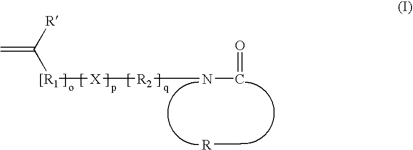 and from 1% to 48% by weight of at least one ionic hydrophilic monomer; and at least one block B obtained from monomers not comprising an ethylenic monomer comprising a lactam ring of formula (I), or comprising a minor proportion thereof; a cosmetic or pharmaceutical composition, such as a hair composition, comprising the copolymer; a cosmetic process for making up or caring for keratin materials, comprising applying to the keratin materials the composition; and other uses of the copolymer.

This application claims benefit of U.S. Provisional Application No. 60/509,539, filed Oct. 9, 2003.

Disclosed herein are novel polymers of specific structure of block ethylenic copolymer type comprising at least one vinyllactam block.

Further disclosed herein is a composition, such as a cosmetic or pharmaceutical composition, for example, a hair composition, comprising these polymers of specific structure.

The present disclosure also relates to the cosmetic use of these polymers for treating keratin materials, such as the skin, the nails and the hair.

As used herein, the term “vinyllactam block” means a block comprising at least one lactam unit chosen, for example, from lactam derivatives; in other words, in general, a block that can be prepared by polymerization of a monomer comprising a lactam ring.

Many cosmetic compositions, such as hair compositions, referred to as “hair styling” compositions, which are in the form of aerosols (“sprays”), gels, mousses or shampoos, contain resins or polymers. Such polymers are, for example, acrylic polymers with high glass transition temperatures (Tg), such as those described in document FR-A-2 439 798. These polymers can provide, for example in terms of styling, hold of the head of hair, but they may have the drawback of being excessively brittle, which does not allow good hold of the head of hair over time.

In the case of varnishes for nails, such polymers are typically not resistant to impacts.

To solve the problems posed by these polymers, plasticizers are added to cosmetic compositions in order to reduce the glass transition temperature. However, in this case, the polymers may have a tendency to exhibit “tacky” effects or, in the case of styling, to not allow “ultra-strong” fixing.

In addition, when the hair is styled, numerous known hairstyling polymers may form white particles, which may be unacceptable, in particular when the hair is brown and/or thick. Other drawbacks presented by the polymers currently used include their possible incompatibility with the existing aerosol propellants.

Increasingly easy removal by shampooing, with all types of shampoo, is also desired.

Nonionic polymers or copolymers based on vinyllactams, for example, based on vinylpyrrolidone, such as vinylpyrrolidone/vinyl acetate copolymers, are also known in the cosmetics field. These copolymers, however, often exhibit difficulty of removal by shampooing.

The polymers based on vinyllactams may also have high hygroscopicity, which may give them a highly tacky nature in the presence of ambient moisture.

Thus, document EP-A-1 002 811 from BASF describes water-soluble or water-dispersible grafted polymers obtained by free-radical polymerization of essentially acrylic monomers, and of a polymerizable prepolymer based on vinyllactam, for example, based on vinylpyrrolidone or vinylcaprolactam.

These polymers are, for example, used in hair compositions.

U.S. Pat. No. 6,193,961 from ISP describes a homogeneous terpolymer of N-vinyllactam, such as N-vinylpyrrolidone and N-vinylcaprolactam, of a dimethylaminoalkyl acrylate or of dimethylaminoalkylacrylamide and of a polysiloxane monomer.

These terpolymers are used in cosmetic and care compositions, for example, cosmetic compositions such as styling gels, foams, and mousses.

This U.S. patent cites numerous other patents mentioning the use of vinyllactam-based polymers in skincare and haircare compositions, such as U.S. Pat. Nos. 3,914,403, 3,954,960, 4,039,734, 4,057,533, 4,210,161, 4,223,009, 4,586,518, 4,764,363, 4,834,968, 4,842,850, 4,902,499, 4,906,459, 4,923,694, 4,963,348, 5,011,895, 5,015,708, 5,126,124, 5,158,762, 5,275,809, 5,502,136, and documents WO-A-91/15186, WO-A-91/15185, EP-A2-412 704, EP-A1-0 412 707 and JP-A-57 126 409.

In this same document, it is also indicated that many patents disclose the use, for example, of an N-vinyllactam in the field of cosmetics and pharmaceutical products, such as in hair aerosols. These patents are U.S. Pat. Nos. 3,910,862, 4,923,694, 5,045,617, 5,321,110, 5,492,988 and 5,637,296.

U.S. Pat. No. 3,954,960 relates to cosmetic and hair compositions containing, as film-forming resin, a quaternized copolymer of vinylpyrrolidone and of a copolymerizable vinyl monomer, i.e., a dialkylaminoalkyl (meth)acrylate.

U.S. Pat. No. 3,914,403 relates to hair compositions containing as film-forming resin: an N-vinylpyrrolidone homopolymer or copolymer mixed with a quaternized copolymer based on vinylpyrrolidone and on a vinyl monomer that is copolymerizable therewith, i.e., a dialkylaminoalkyl (meth)acrylate.

U.S. Pat. No. 5,502,136 relates to a process for preparing copolymers of vinylpyrrolidone and of vinyl acetate via free-radical polymerization.

Document WO-A-00/68282 relates to terpolymers of vinylpyrrolidone (VP), of dimethylaminopropylmethacrylamide (DMAPMA), and of the DMAPMA derivative quaternized with a C₈ to C₂₄ alkyl chain, and hair and cosmetic compositions comprising the terpolymers.

In such copolymers, fatty chains ae often inserted in order to reduce the tack and to increase the moisture resistance, but the range of properties of the polymers obtained and the choice of monomers that may be used to obtain water-borne polymers may thus be limited. Moreover, no value for the tack of these copolymers is mentioned.

The document by Matyjasezwski et al., Prepr. 38(1), 1997, p. 695, describes copolymers containing an N-vinylpyrrolidone skeleton and PDMS grafts for hydrogels.

Patent WO 97/18247 from the same K. Matyjaszewski describes, on page 103, an example of a vinylpyrrolidone homopolymer.

Document FR-A-2 327 761 relates to cosmetic compositions comprising a polymer resulting from the polymerization, in the presence of cerium, of an unsaturated monomer onto a polyvinylpyrrolidone diol. A PVP-poly(lauryl methacrylate) polymer is mentioned. The use of a vinylpyrrolidone/glucosamine acrylate copolymer is also described. The polymers of this document are very specific polymers, generally of branched structure.

These polymers may give the hairstyle sheen and hold over time; however, they may also give the hairstyle a certain amount of rigidity, causing an unnatural appearance.

Furthermore, these polymers are not water-borne, but are borne in alcohols, or alternatively in water in the presence of surfactants and require the presence of surfactants in the composition to promote their removal by shampooing.

There is thus a need for a polymer, which, when included in a composition, such as a cosmetic composition, is such that this composition does not have at least one of the drawbacks, defects, limitations and disadvantages of the compositions known in the art.

There is, for example, a need for a polymer and a composition comprising the polymer that has an optimum combination of properties in terms of rigidity, “tack”, such as the tacky feel, and of removal by shampooing.

Thus, a hair composition comprising the polymer ideally should make it possible to obtain more hold, while at the same time maintaining a natural look. The polymer should also, in such compositions, show good styling properties and, during disentangling, should not become powdery, i.e., should not form visible “flaking” residues. Moreover, the polymer should be compatible with aerosol propellant gases.

Finally, the polymer, in such hair compositions, should be easily removable by shampooing and, in all cases, should have a better capacity for removal than the polymers known in the art.

In the case of varnishing the nails, the production of a glossy film is desired, and this film furthermore needs to be resistant to mechanical attack. The polymer contained in the formula should thus be capable of showing excellent resistance to mechanical abrasion.

In the case of a skin treatment, the makeup used, which includes the polymer, should adhere to the skin, without pulling on the skin, while at the same time being comfortable (not causing any tautness).

In all the cases and irrespective of the composition in which the polymer is used, this polymer should give a product with a non-tacky feel, for example, as a function of the humidity, and especially under conditions of high humidity (namely, in general for relative humidity (RH) values ranging from 50% to 100%).

In other words, there is a need for a film-forming polymer which has little or no tack and, in any case, a reduced level of tack, in humidity, compared with the film-forming polymers known in the art, and which can also show improved removal by shampooing.

Disclosed herein is a polymer that can satisfy at least one of the needs, criteria and requirements mentioned above and which can solve at least one of the problems of the polymers known in the art.

More specifically, disclosed herein is a linear block ethylenic copolymer comprises:
at least one block A that is obtained from monomers comprising from 52% to 99% by weight of at least one ethylenic monomer comprising a lactam ring, chosen from monomers of formula (I) below: 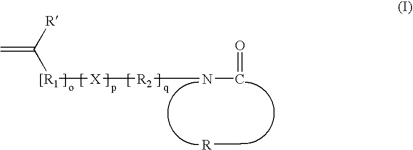
wherein:
   R is a group —(CH₂)ₙ—, wherein n is an integer from 3 to 12, and wherein at least one of the carbon atoms is optionally replaced with an atom chosen from nitrogen and oxygen atoms, and is optionally substituted with at least one alkyl group chosen from C₁ to C₆ alkyl groups;
   R′ is chosen from H and a methyl group;
   R₁ and R₂, which may be identical or different, are each chosen from linear, branched and cyclic C₁-C₂₂ alkylene and aralkylene groups, wherein at least one of the carbon atoms is optionally replaced with an atom chosen from oxygen and nitrogen atoms;
   X is chosen from —OCO—, —NHCO—, —COO— and —O—;
   o, p and q, which may be identical or different, are each 0 or 1;
and from 1% to 48% by weight of at least one ionic hydrophilic monomer;
   and at least one block B that is obtained from monomers not comprising an ethylenic monomer comprising a lactam ring of formula (I), or comprising a minor proportion thereof.

The amount (from 52% to 99%) by weight of the at least one ethylenic monomer comprising a lactam ring and the amount (from 1% to 48%) by weight of the at least one ionic hydrophilic monomer are given relative to the total weight of the monomers from which the block A is obtained. In other words, the amount in units prepared from the at least one ethylenic monomer comprising a lactam ring of the block A is from 52% to 99% by weight and the amount in units prepared from the at least one ionic hydrophilic monomer of the block A is from 1% to 48% by weight.

These amounts by weight, weight contents, are given relative to the total weight of the block A.

In one embodiment, in formula (I), o is O, p is 1, q is 1, R₂ is —CH₂CH₂—, X is chosen from —COO— and —CONH— and R is chosen from —(CH₂)₃—, —(CH₂)₅—, and —(CH₂CH₂NH)—.

The monomer of formula (I) can be chosen, for example, from pyrrilidinoethyl methacrylate, pyrrilidinoethyl acrylate, ureidoethyl acrylate, and ureidoethyl methacrylate.

For example, the block A may be prepared from monomers comprising from 52% to 99% by weight of at least one ethylenic monomer comprising a lactam ring which is a vinyllactam of formula (II) below: 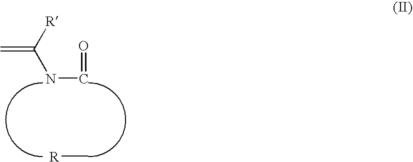
wherein R and R′ have the meaning already given above.

In one embodiment, in formulae (I) and (II) above, R is —(CH₂)ₙ— wherein n is an integer from 3 to 5, or alternatively R is —CH₂—CH₂—NH—.

For example, the N-vinyllactam of formula (II) can be chosen from N-vinylpyrrolidone (n=3), N-vinylpiperidinone (valerolactam) (n=4), N-vinylcaprolactam (n==5), N-vinylimidazolidinone wherein R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

The N-vinyllactams include, for example, N-vinylpyrrolidone, N-vinylcaprolactam and vinylimidazolidinone.

Further disclosed herein are cosmetic or pharmaceutical compositions comprising the linear block ethylenic copolymers.

When they are incorporated into such compositions, the copolymers having the specific structure as disclosed herein make it possible to obtain highly advantageous properties, or a combination of highly advantageous properties, which was not able to obtain with the polymers known in the art.

In general, the copolymers as disclosed herein can have, because of their particular structure, reduced hygroscopicity when compared with the copolymers known in the art comprising vinyllactam units.

The copolymers as disclosed herein can have an optimum combination of rigidity and non-tacky nature and they can thus lead to compositions or systems having, for example, enhanced mechanical strength, wear strength and hold over time, and reduced fragility, while at the same time not being tacky.

The copolymers as disclosed herein can also have the property of being easily removable by shampooing, and, in all cases, more easily than the polymers known in the art.

The copolymers as disclosed herein, which are, for example, film-forming polymers, may thus be defined as polymers that have little or no “tack”

Thus, when the copolymers as disclosed herein are used in compositions for treating the hair, such as lacquers, they can give greater hold over time. They can be less fragile than a standard lacquer and at the same time not be tacky. The phenomenon of powdering on the hair, which is observed during disentangling with the compositions known in the art, can be avoided.

Furthermore, these compositions may be easily removed by shampooing and, in any case, more easily than compositions comprising the polymers known in the art.

In the case of nail varnishes, the varnish comprising the copolymer as disclosed herein can have greater wear strength and is not tacky, while at the same time adhering to the nail. As a result, the loss of gloss, i.e., the matting of the film by mechanical marking or by marking with dust which may take place with tacky films known in the art, does not take place with the varnishes and films comprising the copolymer as disclosed herein. The reason for this is that the absence of tack from these copolymers can have the effect that they are not mechanically marked, they do not retain dust and they therefore do not undergo changes in or loss of gloss.

The varnishes comprising the copolymer as disclosed herein, by virtue of the specific structure of the copolymer, can give films which do not chip and which, surprisingly, are not tacky.

In makeup products, for instance, lipsticks and foundations, comprising the copolymer as disclosed herein, the makeup can show good staying power on the lips or the skin, without giving a tacky sensation.

Further disclosed herein is a cosmetic process for making up or caring for keratin materials, comprising applying to the keratin materials the composition as disclosed herein.

The present disclosure also relates to the use of the copolymers as disclosed herein to improve the hairstyle hold, without tack, such as without a tacky feel, of a hair lacquer; the use of the copolymers as disclosed herein to improve the removal by shampooing of a hair composition, such as a hair lacquer; the use of the copolymers disclosed herein to improve the adhesion and the wear resistance, without tack, such as without a tacky feel, of a nail varnish; the use of the copolymers as disclosed herein to optimize the adhesion to the skin, and the comfort of a makeup composition; the use of the copolymers as disclosed herein to reduce the tack, such as the tacky feel, of a cosmetic composition, such as under conditions of high humidity, for example, conditions with relative humidity (RH) values ranging from 50% to 100%; and the use of the copolymers disclosed herein in a cosmetic composition such as a makeup composition for masking wrinkles, which can give the skin a smoothened appearance, without tautness.

The copolymers as disclosed herein can thus provide a solution to at least one of the problems posed by the polymers known in the art.

The unexpected advantageous properties of the copolymers disclosed herein, which are fundamentally linear copolymers, are believed to arise from the specific nature of the blocks of which they are made, and, for example, of the at least one block A comprising from 52% to 99% by weight of the at least one ethylenic monomer comprising a lactam ring, such as a vinyllactam.

By virtue, so it would seem, of the presence in the at least one block A comprising from 52% to 99% by weight of the at least one ethylenic monomer comprising a lactam ring, such as a vinyllactam, and of at least one other monomer (from 1% to 48% by weight), which is chosen from ionic hydrophilic monomers, the copolymers disclosed herein, surprisingly, can be much more easily removed by shampooing than the vinyllactam-based polymers known in the art, while at the same time having reduced hygroscopicity when compared with the vinyllactam-based polymers known in the art, for example, when compared with the nonionic vinyllactam copolymers known in the art.

Similarly, the copolymers as disclosed herein can be, surprisingly, more readily removed by shampooing and have reduced hygroscopicity (attested by lower tack values at high humidity, for example, for RH values ranging from 50% to 100%) when compared with the copolymers known in the art having a block structure formed of blocks comprising vinyllactam homopolymers (for example, polyvinylpyrrolidone, PVP) and blocks prepared from non-hydrophilic monomers, such as methyl methacrylate.

In fact, it turned out, surprisingly, that the incorporation of at least one hydrophilic monomer, such as at least one ionic hydrophilic monomer, directly within the vinyllactam block, which is thus, as disclosed herein, such as in the form of a copolymer (whether an alternating, gradient or random copolymer) of the ethylenic monomer comprising a lactam ring, such as a vinyllactam, and of the ionic hydrophilic monomer, can lead to a significant improvement in their removal by shampooing compared with an analogous copolymer, in which polyvinyllactam blocks consist of vinyllactam homopolymers and therefore do not comprise ionic hydrophilic monomers. This advantage and this improvement are linked to the fact that the block is a copolymer and are present irrespective of the type of copolymer, random, alternating or gradient.

Similarly, the removal by shampooing of the copolymers as disclosed herein can be greatly improved compared with copolymers known in the art prepared from vinyllactams and from a nonionic hydrophilic monomer. Surprisingly, the specific choice of a hydrophilic ionic monomer can ensure excellent removal by shampooing, whereas the copolymers prepared from vinyllactams and from a nonionic hydrophilic monomer, such as those described in document FR-A-2 327 761, may be removed with difficulty, or even not at all, and the compositions comprising these copolymers with a nonionic monomer require the incorporation of an ionic surfactant into the composition to facilitate or ensure the removal by shampooing.

Among the ionic hydrophilic monomers, it was found that the effect of improving the removal by shampooing could be more enhanced with anionic hydrophilic monomers.

The copolymer as disclosed herein also comprises at least one block B, free of any ethylenic monomer comprising a lactam ring, such as a vinyllactam, or in which the ethylenic monomer comprising a lactam ring, such as a vinyllactam, is present in a minor amount. The copolymers disclosed herein thus differ from some copolymers known in the art that are random copolymers of vinyllactam, for example, of vinylpyrrolidone and of another monomer, for example, a hydrophobic monomer, and which therefore do not comprise the block B as disclosed herein.

Such random copolymers known in the art, which are described, for example, in document WO-A-00/68282 mentioned above, can be difficult to remove by shampooing, owing to the presence of hydrophobic units. In contrast, the copolymers disclosed herein can have varied structures because of their block structure with at least one block B and can thus lead to properties that are adapted to the application.

The at least one block B may be chosen as desired to give the copolymer according to the present disclosure all the intended desirable properties—besides the non-tacky nature—that correspond to specific applications. For example, the at least one block B may be chosen so as to control the flexibility of the copolymer and to obtain, surprisingly, copolymers that are not fragile and brittle, and that can give chip-free films, while at the same time not being tacky, for example, not having a tacky feel, and being very readily removable by shampooing.

If it is arranged such that the at least one block B is hydrophilic, then the excellent capacity to be removed by shampooing of the copolymers as disclosed herein can be further enhanced.

Nothing known in the art suggests that by using a linear copolymer and by stipulating:
that at least one block A of this copolymer comprises an amount ranging from 52% to 99% by weight of at least one ethylenic monomer comprising a lactam ring, such as a vinyllactam;
that at least one other monomer of this block A is hydrophilic such as ionic hydrophilic; and,
that at least one other block B has a structure different from the block A and is free of or comprises in minor amount of the ethylenic monomer comprising a lactam ring, such as an N-vinyllactam,
   it would be possible to achieve, as disclosed herein, an improvement in the removal by shampooing of the copolymer or of a composition comprising the copolymer, an unprecedented reduction in the tacky nature of the copolymer and to obtain, by selecting the desired block B, a combination of excellent properties for this copolymer.

The specific structure of the copolymer as disclosed herein can lead to optimization of its properties, leading to an excellent equilibrium between, for example, the properties in terms of mechanical strength, the absence of tack, such as the tack-free feel, and the ease of removal by shampooing.

Without wishing to be bound by any theory, the advantageous properties of the copolymer according to the present disclosure are thought to arise from the fact that the nature of the blocks is specifically chosen so as to promote the separation of the phases from each other and thus, inter alia, to give optimum control of the rigidity and the hygroscopicity and thus of the tack of the copolymer, and also the capacity of the copolymer to be removed by shampooing.

Moreover, the fact that the polymer is linear entails a much simpler and much more controlled synthesis, which makes it possible to precisely predict the structure of the polymers obtained, and thus to optimize the final properties of the polymers.

More specifically, the copolymers disclosed herein are block copolymers. This term generally means that the copolymers comprise blocks that are covalently attached to each other.

In addition, two successive blocks are of different nature. In contrast, two non-successive blocks A or B may be of the same nature. Each block A comprises a copolymer of the at least one ethylenic monomer comprising a lactam ring, such as a vinyllactam, and of the at least one ionic hydrophilic monomer, which generates a random, alternating or gradient copolymer.

The block B comprises a homopolymer or a copolymer, wherein it is possible for the copolymer in turn to be a random copolymer, an alternating copolymer or a gradient copolymer.

The polymer as disclosed herein may also comprise at least one more block, such as another block C other than the blocks A and B and optionally yet other blocks, for example, a block D other than A, B and C.

The polymer disclosed herein may thus be chosen from diblock copolymers of AB type or triblock copolymers of ABA, BAB, ABC or ACB type, with C other than A or B.

The polymer as disclosed herein may also be chosen from multiblock copolymers comprising more than three identical or different blocks of (AB)ₙ, (ABA)ₙ, (BAB)ₙ, (ABC)ₙ or (ACB)ₙ type, with C other than A or B, or multiblock copolymers comprising more than three different blocks of ABCD type.

In general, as has already been mentioned, the nature of the blocks is chosen so as to promote the phase separation between the blocks, since this can predetermine the properties.

The nature and number of the blocks are chosen by a person skilled in the art as a function of the desired properties within the limits of the conditions specified above for blocks A and B.

The copolymers disclosed herein are defined as being ethylenic copolymers. This means that the monomers from which the blocks constituting the copolymer are derived are monomers comprising a carbon-carbon unsaturated double bond of ethylenic type.

In addition, the copolymer disclosed herein is a linear copolymer. This means that the present disclosure is not intended to cover copolymers having a non-linear structure, for example, a branched, starburst, grafted or other structure. The linear nature of the copolymers as disclosed herein is important for giving the compositions comprising the copolymers the advantageous properties described above.

In one embodiment, the copolymer is a film-forming polymer, i.e., it is capable by itself, or in the presence of an auxiliary film-forming agent, at a temperature ranging from 20° C. to 30° C., of forming a continuous film (viewed by the naked eye) and adhering to a keratin support.

As disclosed herein, the copolymer comprises at least one block A that is obtained from monomers comprising from 52% to 99% by weight of the at least one ethylenic monomer comprising a lactam ring, for example a vinyllactam, chosen from those of formula (I) and, for example, those of formula (II).

The amount (from 52% to 99%) by weight of the at least one ethylenic monomer comprising a lactam ring and the amount (from 1% to 48%) by weight of the at least one ionic hydrophilic monomer are given relative to the total weight of the monomers from which the block A is obtained. In other words, the amount in units prepared from the at least one ethylenic monomer comprising a lactam ring of the block A is from 52% to 99% by weight and the amount in units prepared from the at least one ionic hydrophilic monomer of the block A is from 1 to 48% by weight.

These amounts by weight, weight contents, are given relative to the total weight of the block A.

The percentage of the at least one ethylenic monomer comprising a lactam ring, such as a vinyllactam of formula (I) or (II), in the monomers from which the block A is obtained is from 52% to 99%, such as from 55% to 95% by weight, further such as from 60% to 80% by weight, even further such as from 65% to 75% by weight; and in one embodiment, it is 70% by weight.

In other words, the amount, content, (by weight) in units prepared from the at least one ethylenic monomer comprising a lactam ring, such as vinyllactam of the block A is from 52% to 99% by weight. And, the amount (by weight) in units prepared from the at least one ethylenic monomer comprising a lactam ring of the block A may range, for example, from 55% to 95% by weight, such as from 60% to 80% by weight, and further such as from 65% to 75% by weight, and may also be, for example, 70% by weight. These amounts are given relative to the total weight of the block A.

The block A comprising from 52% to 99% by weight of the at least one ethylenic monomer comprising a lactam ring, such as a vinyllactam, may have any overall glass transition temperature (Tg) for the block, but it generally has a “high” overall glass transition temperature.

The term “high” generally means that this Tg may be from 0 to 250° C., such as from 0 to 220° C. and further such as from 5 to 200° C.

The block A may only contain hydrophilic monomers.

As used herein, a hydrophilic block means a water-soluble or water-dispersible block.

The block A may, for example, be a hydrophilic block.

However, the block A may have a hydrophobic nature depending on the neutralizer used to neutralize the ionic function.

The term “hydrophilic” is defined above, meaning water-soluble or water-dispersible.

The at least one ethylenic monomer comprising a lactam ring, such as a vinyllactam, may be chosen from N-vinyllactams and derivatives thereof, which may comprise, for example, at least one alkyl substituent chosen from C₁ to C₆ alkyl substituents, such as methyl, ethyl, n-propyl, isopropyl, n-butyl and sec-butyl.

In one embodiment, the at least one ethylenic monomer comprising a lactam ring, such as an N-vinyllactam, of formula (I) is chosen from pyrrilidinoethyl acrylate and pyrrilidinoethyl methacrylate.

For example, the N-vinyllactam of formula (II) may be chosen from N-vinylpyrrolidone (n=3), N-vinylpiperidinone (valerolactam) (n=4), N-vinylcaprolactam (n=5), N-vinylimidazolidinone wherein R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

The N-vinyllactams may be chosen, for example, from N-vinylpyrrolidone, N-vinylcaprolactam and vinylimidazolidinone.

As disclosed herein, the block A, comprising from 52% to 99% by weight of the at least one ethylenic monomer comprising a lactam ring, such as an N-vinyllactam, is a copolymer, the monomers from which it is obtained thus comprise, besides the at least one ethylenic monomer comprising a lactam ring, such as a vinyllactam, of formula (I), at least one other monomer, which may be the same or different, and is chosen from ionic hydrophilic monomers.

The monomers from which the block A is obtained comprise the at least one ionic hydrophilic monomer in an amount ranging from 1% to 48% by weight relative to the total weight of the monomers from which the block A is obtained. In one embodiment, the at least one ionic hydrophilic monomer is present in the monomers from which the block A is obtained in an amount ranging from 5% to 45% by weight, such as from 20% to 40% by weight, further such as from 25% to 35% by weight, and, for example, being 30% by weight. In other words, the amount (by weight) of units prepared from the at least one ionic hydrophilic monomer of the block A is from 1% to 48% by weight. And generally, the amount (by weight) in units prepared from the at least one ionic hydrophilic monomer may range from 5% to 45% by weight, such as from 20% to 40% by weight, further such as from 25% to 35% by weight, and, for example, be 30% by weight. These amounts are given relative to the total weight of the block A.

The monomers of the block A, other than the at least one ethylenic monomer comprising a lactam ring, such as vinyllactam, are chosen from ethylenic monomers that are copolymerizable with the lactam derivative monomer, irrespective of the coefficient of reactivity thereof.

The block A, generally represents 50% by weight or more of the total weight of the copolymer as disclosed herein, such as from 50% to 99%, further such as from 55% to 95%, and even further such as from 60% to 90% by weight relative to the total weight of the copolymer.

The at least one block B is obtained from at least one ethylenic monomer generally chosen from: allylic monomers, acrylates, methacrylates, acrylamides, methacrylamides and vinyl monomers, and mixtures thereof, and optionally ethylenic monomers comprising a lactam ring, for example, vinyllactam monomers, of formula (I) or (II), wherein the ethylenic monomers comprising a lactam ring, for example, vinyllactams, of formula (I) or (II), is in a minor proportion, i.e., of less than 50% by weight, such as less than or equal to 45% by weight, further such as less than or equal to 40% by weight and even further such as less than or equal to 30% by weight relative to the total weight of the at least one block B.

The at least one block B may be hydrophobic or hydrophilic. In one embodiment, the at least one block B is hydrophilic, which can further increase the ease of removal by shampooing.

For example, the at least one block B may have a low glass transition temperature Tg, i.e., for example, less than or equal to 50° C.

In one embodiment, the number-average molecular mass of each block, whether it is the block A or the block B, is from 2000 to 1 000 000, such as from 2000 to 800 000 and further such as from 2000 to 500 000.

The number-average molecular mass of the overall copolymer (for example, A-b-B) is generally from 4000 to 1 000 000, such as from 4000 to 800 000 and further such as from 4000 to 500 000.

In one embodiment, the at least one block B is present in an amount ranging from 1% to 50% by weight of the total weight of the copolymer, such as from 5% to 45% by weight and further such as from 10% to 40% by weight of the total weight of the copolymer.

The copolymer as disclosed herein will now be described more in detail.

Since the glass transition temperature Tg is an important parameter for defining the blocks of the copolymer as disclosed herein, such as the at least one block B of the copolymer disclosed herein and, consequently, the copolymer as disclosed herein, it is important to point out that the glass transition temperatures of the blocks of the copolymers used herein are measured by differential thermal analysis, “Differential Scanning Calorimetry” (DSC) for the dry polymer, at a heating rate of 10° C./minute.

Each block B of the copolymer as disclosed herein is derived from one type of monomer or from several different types of monomers.

This means that each block B may comprise at least one polymer chosen from homopolymers and copolymers; the copolymers which constitute the block B may in turn be random, alternating and gradient copolymers.

Each block A of the copolymer as disclosed herein comprises a copolymer which may be chosen from alternating, random and gradient copolymers; wherein this copolymer comprises a proportion ranging from 52% to 99% by weight of the at least one ethylenic monomer comprising a lactam ring, such as a vinyllactam.

Each of the blocks A and B constituting the copolymer as disclosed herein are now described in detail.

As disclosed herein, the block A, comprising from 52% to 99% by weight of the at least one ethylenic monomer comprising a lactam ring, such as a vinyllactam, further comprises from 1% to 48% by weight of at least one ionic hydrophilic monomer.

The term “hydrophilic monomer” means that the homopolymer prepared from this monomer is water-soluble or water-dispersible, or that it may be made water-soluble or water-dispersible following a treatment such as hydrolysis.

The at least one ionic hydrophilic monomer is thus chosen, for example, from anionic and cationic monomers and betaines, as described below.

The at least one ionic hydrophilic monomer can also be chosen from monomers that may be made hydrophilic (ionic) following hydrolysis; for example, the at least one ionic hydrophilic monomer is chosen from acid-hydrolysable monomers of (meth)acrylic ester type.

A homopolymer is water-soluble, if it is soluble in water, to a proportion of at least 5% by weight at 25° C. relative to the total weight of the homopolymer.

A homopolymer is water-dispersible if it forms, at a concentration of 5% by weight, at 25° C., a stable suspension of generally spherical fine particles. The mean size of the particles constituting the dispersion is less than 1 μm and for example, ranges from 5 to 400 nm and such as from 10 to 250 nm. These particle sizes are measured by light scattering.

The at least one ionic hydrophilic monomer, from which the block A is prepared, can, for example, be chosen from cationic monomers and anionic monomers.
Examples of the cationic monomers include:
2-vinylpyridine (Tg: 104° C.);
4-vinylpyridine (Tg: 142° C.);
dimethylaminoethyl (meth)acrylate;
diethylaminoethyl (meth)acrylate;
dimethylaminopropyl (meth)acrylamide; and
salified and quaternized forms thereof, whether they are salts of mineral acids, such as sulfuric acid and hydrochloric acid, or salts of organic acids.

The organic acids may comprise at least one acid group chosen from carboxylic, sulfonic and phosphonic acid groups. They may be linear, branched or cyclic aliphatic acids or alternatively aromatic acids. These acids may also comprise at least one hetero atom chosen from O and N, for example, in the form of hydroxyl groups.

An example of an acid comprising an alkyl group is acetic acid, CH₃COOH.

An example of a polyacid is terephthalic acid.

Examples of hydroxy acids include citric acid and tartaric acid.

Examples of the anionic monomers include:
acrylic acid, methacrylic acid, crotonic acid, maleic anhydride, itaconic acid, fumaric acid, maleic acid and vinylbenzoic acid;
styrenesulfonic acid, 2-acrylamido-2-methylpropanesulfonic acid and vinylsulfonic acid,
vinylphosphoric acid and sulfopropyl methacrylate,
   and the salts thereof.

The neutralizer may be a mineral base, such as LiOH, NaOH, KOH, Ca(OH)₂ and NH₄OH; or an organic base. It may be chosen from primary, secondary and tertiary alkylamines, such as triethylamine and butylamine. The primary, secondary and tertiary alkylamines may comprise at least one atom chosen from nitrogen and oxygen atoms and may thus comprise, for example, an alcohol function, for example, 2-amino-2-methylpropanol and triethanolamine.
The monomers that may be hydrophilic following hydrolysis may be chosen, for example, from monomers of (meth)acrylic ester type that can be hydrolyzed to acids, such as ethyl, tert-butyl and benzyl (meth)acrylates.

The hydrolysis will be performed once the polymer has been synthesized, under acidic conditions (such as sulfuric acid, hydrochloric acid and trifluoroacetic acid) or under basic conditions (in the presence of alkaline-earth metal hydroxides such as sodium hydroxide and potassium hydroxide, alkali metal alkoxides such as potassium t-butoxide, and amines such as aqueous ammonia). The hydrolysis generally takes place at a temperature ranging from 5 to 100° C., such as from 15 to 80° C.

The polymer will then be purified by repeated precipitations.

In one embodiment, anionic monomers are used since they can lead to an even higher improvement in the removal by shampooing.

The at least one ionic hydrophilic monomers may be chosen, for example, from:
acrylic acid, methacrylic acid, crotonic acid, maleic anhydride, vinylbenzoic acid, styrenesulfonic acid, 2-acrylamido-2-methylpropanesulfonic acid and vinylsulfonic acid, and
tert-butyl acrylate and ethyl acrylate followed by hydrolysis.

The copolymer as disclosed herein may also comprise at least one block B that is obtained from at least one monomer not comprising an ethylenic monomer comprising a lactam ring, such as a vinyllactam, of formula (I) or comprising a minor proportion thereof. There is no limitation as to the nature of the monomers that are capable of forming the block B, with the exception, of course, of the limitation as to the amount of ethylenic monomer comprising a lactam ring, such as a vinyllactam, that may be used for the preparation of this block B.

The at least one monomer may be hydrophobic, non-hydrophilic, but is may also be hydrophilic. The term “hydrophilic” has already been defined above.

The at least one monomer from which the at least one block B is obtained may be chosen from a wide variety of monomers, the Tg values of these monomers may have any value, and these monomers may also be hydrophilic or hydrophobic.

The at least one monomer from which the at least one block B is obtained may thus be chosen, for example, from the monomers described below:

C₂-C₁₀ ethylenic hydrocarbons, such as ethylene, isoprene and butadiene;

acrylates of formula CH₂═CHCOOR₃, and methacrylates of formula:

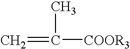

wherein R₃ is chosen from:
linear and branched C₁-C₁₈ alkyl groups in which at least one hetero atom chosen from O, N, S and P is optionally intercalated,
wherein at least one of the alkyl groups is also optionally substituted with at least one substituent chosen from hydroxyl groups, halogen atoms (Cl, Br, I and F) and groups Si(R₄R₅), wherein R₄ and R₅, which may be identical or different, are each chosen from C₁ to C₆ alkyl groups and a phenyl group,

examples of these alkyl groups include methyl, ethyl, propyl, butyl, isobutyl, tert-butyl, ethylhexyl, octyl, lauryl and stearyl,

examples of these alkyl-based groups include C₁₋₄ hydroxyalkyl groups such as 2-hydroxyethyl and 2-hydroxypropyl, (C₁₋₄)alkoxy(C₁₋₄)alkyl groups such as methoxyethyl, ethoxyethyl and methoxypropyl,
C₃ to C₁₂ cycloalkyl groups, such as an isobornyl group,
C₃ to C₂₀ aryl groups such as a phenyl group,
C₄ to C₃₀ aralkyl groups (C₁ to C₈ alkyl group) such as 2-phenylethyl and benzyl,
4- to 12-membered heterocyclic groups comprising at least one hetero atom chosen from O, N and S, wherein the ring is aromatic or non-aromatic,
heterocyclylalkyl groups (C₁ to C₄ alkyl group), such as furfurylmethyl and tetrahydrofurfurylmethyl,
   wherein at least one of the cycloalkyl, aryl, aralkyl, heterocyclic and heterocyclylalkyl groups is optionally substituted with at least one substituent chosen from hydroxyl groups, halogen atoms and linear and branched C₁ to C₄ alkyl groups in which at least one hetero atom chosen from O, N, S and P is optionally intercalated, at least one of the alkyl groups is optionally substituted with at least one substituent chosen from hydroxyl groups, halogen atoms (Cl, Br, I and F) and groups Si(R₄R₅), wherein R₄ and R₅, which may be identical or different, are each chosen from C₁ to C₆ alkyl groups and a phenyl group,
examples of the group R₃ include methyl, ethyl, propyl, isobutyl, n-butyl, tert-butyl, hexyl, ethylhexyl, octyl, lauryl, isooctyl, isodecyl, dodecyl, cyclohexyl, t-butylcyclohexyl, t-butylbenzyl, isobornyl, phenyl, furfurylmethyl, tetrahydrofurfuryl-methyl, 2-hydroxyethyl, 2-hydroxypropyl, 2-hydroxybutyl, methoxyethyl, ethoxyethyl, methoxyethyl, methoxypropyl and 2-ethylperfluorohexyl groups,
another example of the group R₃ for the acrylates is the group —(OC₂H₄)ₘ—OR″, with m=5 to 150 and R″ is chosen from H and C₁ to C₃₀ alkyl groups, for example, —POE-methoxy and —POE-behenyl;
(meth)acrylamides of formula: 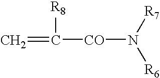
wherein:
R₈ is chosen from H and a methyl group; and
R₆ and R₇, which may be identical or different, are each chosen from a hydrogen atom and linear and branched C₁-C₁₈ alkyl groups, in which at least one hetero atom chosen from O, N, S and P is optionally intercalated, at least one of the alkyl groups is also optionally substituted with at least one substituent chosen from hydroxyl groups, halogen atoms (Cl, Br, I and F) and groups Si(R₄R₅), wherein R₄ and R₅, which may be identical or different, are each chosen from C₁ to C₆ alkyl groups and a phenyl group,
   examples of these groups include methyl, ethyl, n-butyl, t-butyl, isopropyl, isohexyl, isooctyl, isononyl and C₁₋₄ hydroxyalkyl groups, such as 2-hydroxyethyl,
C₃ to C₁₂ cycloalkyl groups, such as an isobornyl group,
C₃ to C₂₀ aryl groups such as phenyl,
C₄ to C₃₀ aralkyl groups (C₁ to C₈ alkyl group) such as 2-phenylethyl and benzyl,
4- to 12-membered heterocyclic groups comprising at least one hetero atom chosen from O, N and S, wherein the ring is aromatic or non-aromatic,
heterocyclylalkyl groups (C₁ to C₄ alkyl group), such as furfurylmethyl and tetrahydrofurfurylmethyl,
   wherein at least one of the cycloalkyl, aryl, aralkyl, heterocyclic and heterocyclylalkyl groups is optionally substituted with at least one substituent chosen from hydroxyl groups, halogen atoms and linear and branched C₁ to C₄ alkyl groups in which at least one hetero atom chosen from O, N, S and P is optionally intercalated, at least one of the alkyl groups is also optionally substituted with at least one substituent chosen from hydroxyl groups, halogen atoms (Cl, Br, I and F) and groups Si(R₄R₅), wherein R₄ and R₅, which may be identical or different, are each chosen from C₁ to C₆ alkyl groups and a phenyl group.

Examples of the (meth)acrylamide monomers include (meth)acrylamide, N-ethyl(meth)acrylamide, N-butylacrylamide, N-t-butylacrylamide, N-isopropylacrylamide, —N,N-dimethyl(meth)acrylamide, N,N-dibutylacrylamide, N-octylacrylamide, N-dodecylacrylamide, undecylacrylamide and N(2-hydroxypropyl methacrylamide);

the allylic compounds of formula:
CH₂═CH—CH₂—R₉ or CH₂═C(CH₃)—CH₂—R₉; and

the vinyl compounds of formula:
CH₂═CH—R₉,

wherein R₉ is a group chosen from:

hydroxyl,

—Cl,

—NH₂,

—OR₁₀, wherein R₁₀ is chosen from a phenyl group and C₁ to C₁₂ alkyl groups (the monomer is a vinyl ether or an allyl ether),

acetamide: —NHCOCH₃,

—OCOR₁₁, wherein R₁₁ is chosen from:
linear and branched C₂-C₁₂ alkyl groups (the monomer is a vinyl ester or an allylic ester),
C₃ to C₁₂ cycloalkyl groups such as isobornyl and cyclohexyl,
C₃ to C₂₀ aryl groups such as phenyl,
C₄ to C₃₀ aralkyl groups (C₁ to C₈ alkyl group) such as 2-phenylethyl and benzyl,
4- to 12-membered heterocyclic groups comprising at least one hetero atom chosen from O, N and S, wherein the ring is aromatic or non-aromatic,
heterocyclylalkyl groups (C₁ to C₄ alkyl group), such as furfurylmethyl and tetrahydrofurfurylmethyl,
   wherein at least one of the cycloalkyl, aryl, aralkyl, heterocyclic and heterocyclylalkyl groups is optionally substituted with at least one substituent chosen from hydroxyl groups, halogen atoms and linear and branched C₁ to C₄ alkyl groups in which at least one hetero atom chosen from O, N, S and P is optionally intercalated, at least one of the alkyl groups is also optionally substituted with at least one substituent chosen from hydroxyl groups, halogen atoms (Cl, Br, I and F) and groups Si(R₄R₅), wherein R₄ and R₅, which may be identical or different, are each chosen from C₁ to C₆ alkyl groups and a phenyl group.

Examples of vinyl monomers include vinylcyclohexane and styrene.

Examples of vinyl esters include: vinyl acetate, vinyl propionate, vinyl butyrate, vinyl ethylhexanoate, vinyl neononanoate and vinyl neododecanoate. Among the vinyl ethers, examples include methyl vinyl ether, ethyl vinyl ether and isobutyl vinyl ether;
(meth)acrylic and (meth)acrylamide and vinyl monomers comprising at least one group chosen from fluoro and perfluoro groups, such as ethylperfluorooctyl methacrylate; and
silicone (meth)acrylic and vinyl monomers, such as methacryloxypropyltris(trimethylsiloxy)silane, acryloxypropylpolydimethylsiloxane and silicone (meth)acrylamides.

The at least one monomer from which the block B is obtained is, for example, chosen from monomers of which the glass transition temperature of the corresponding homopolymer is less than or equal to 110° C., such as less than or equal to 50° C. and further such as less than or equal to 20° C.

The monomers that are, for example, used include those for which the glass transition temperature Tg of the corresponding homopolymer is less than or equal to 50° C., such as: methyl acrylate (Tg=10° C.), ethyl acrylate (Tg=−24° C.), n-butyl acrylate (Tg=−54° C.), t-butyl acrylate (Tg=43° C.), 2-ethylhexyl acrylate (Tg=−50° C.), isobutyl acrylate (Tg=−24° C.), methoxyethyl acrylate (Tg=−33° C.), butyl methacrylate (Tg=20° C.), ethoxyethyl methacrylate (Tg=0° C.), POE methacrylate (n=8 to 10) (Tg=−55° C.) and vinyl acetate (Tg=23° C.); and other monomers, such as methyl methacrylate (Tg=105° C.), ethyl methacrylate, isobutyl methacrylate, furfuryl acrylate, isobornyl acrylate, tert-butylcyclohexyl acrylate, styrene and vinylcyclohexane.

The at least one monomer from which the block B is obtained may be chosen from hydrophilic monomers, including ionic monomers such as cationic monomers, anionic monomers and betaines; nonionic monomers; and monomers that may be made hydrophilic following hydrolysis.

Examples of anionic and cationic monomers have already been given above in the context of the description of the block A.

Examples of ionic monomers of betaine type include:
ethylenic carboxybetaines and sulfobetaines obtained, for example, by quaternization of ethylenically unsaturated monomers comprising an amine function with carboxylic acid sodium salts comprising a labile halogen, for example, sodium chloroacetate, or with cyclic sulfones, for example, propane sulfone.

Examples of nonionic monomers include:
hydroxyalkyl (meth)acrylates, the alkyl group of which comprises from 2 to 4 carbon atoms, for example, hydroxyethyl (meth)acrylate;
vinyllactams;
(meth)acrylamides and (C₁-C₄ N-alkyl)(meth)acrylamides, for instance isobutylacrylamide; and
polysaccharide (meth)acrylates, for instance sucrose acrylate.

However, as disclosed herein, the vinyllactams should constitute only a minor proportion of the block B.

It should be noted that even though the copolymer comprises a hydrophilic block, the overall copolymer is not necessarily hydrophilic.

The linear block ethylenic copolymers as disclosed herein are chosen, for example, from:
diblock copolymers (AB);
triblock copolymers (ABA, BAB, ABC and ACB), with C other than A or B;
multiblock copolymers comprising more than three blocks: (AB)ₙ, (ABA)ₙ, (BAB)ₙ, (ABC)ₙ or (ACB)ₙ, with C other than A or B, or multiblock copolymers comprising more than three different blocks, of ABCD type.

The copolymers as disclosed herein may be prepared by anionic polymerization.

For example, however, the copolymers as disclosed herein are obtained in a first mode by controlled free-radical polymerization and, in one embodiment, the copolymers as disclosed herein may be obtained via a particular “ATRP” polymerization, i.e., the technique known as “Reverse ATRP”, or via the RAFT technique, but the polymers as disclosed herein may, according to a second mode, also be obtained via standard free-radical polymerization.

First Mode

The block copolymers as disclosed herein are, for example, obtained by controlled free-radical polymerization, described, for example, in “New Method of Polymer Synthesis”, Blackie Academic Professional, London, 1995, Volume 2, page 1.

Controlled free-radical polymerization makes it possible to reduce the deactivation reactions of the growing free-radical species, such as the termination step, these being reactions which, in standard free-radical polymerization, irreversibly and uncontrollably stop the growth of the polymer chain.

In order to reduce the probability of the termination reactions, it has been proposed to temporarily and reversibly block the growing free-radical species, by forming “dormant” active species in the form of a bond with a low dissociation energy.

Thus, the polymerization may be performed according to the Atom Transfer Radical Polymerization (ATRP) technique, or by reaction with a nitroxide, or alternatively according to the “reversible addition-fragmentation chain transfer” (RAFT) technique, or, finally, by the “reverse ATRP” technique.

The atom transfer radical polymerization technique, also known as ATRP, comprises blocking the growing free-radical species in the form of a bond of C-halide type (in the presence of a metal/ligand complex). This type of polymerization is reflected by control of the mass of the polymers formed and by a low dispersity index of the masses.

In general, the atom transfer radical polymerization is performed by polymerization of at least one free-radical-polymerizable monomer, in the presence of:
an initiator comprising at least one transferable halogen atom;
a halogenated compound comprising a transition metal capable of participating in a reduction step with the initiator and a “dormant” polymer chain, this compound is referred to as the “chain-transfer agent”; and
a ligand that may be chosen from compounds comprising at least one atom chosen from nitrogen (N), oxygen (O), phosphorus (P) and sulfur (S) atoms, capable of coordinating via a a bond to the compound comprising a transition metal, wherein the formation of direct bonds between the compound comprising a transition metal and the polymer under formation is avoided.

The halogen atom is chosen, for example, from chlorine and bromine atoms.

This process is described, for example, in WO 97/18247 and in the article by Matyjasezwski et al. published in JACS, 117, page 5614 (1995).

The technique of free-radical polymerization by reaction with a nitroxide comprises blocking the growing free-radical species in the form of a bond of C—O—NRₐR_{b} type, wherein Rₐ and R_{b}, which may be identical or different, are each chosen from alkyl radicals comprising from 2 to 30 carbon atoms, or together form, with the nitrogen atom, a ring comprising from 4 to 20 carbon atoms, for instance a 2,2,6,6-tetramethylpiperidyl ring. This polymerization technique is, for example, described in the articles “Living free radical polymerization: a unique technique for preparation of controlled macromolecular architectures”, C J Hawker; Chem. Res., 1997, 30, 373-82 and “Macromolecular engineering via living free radical polymerizations” published in Macromol. Chem. Phys. 1998, Vol. 199, pages 923-935, or alternatively in patent application WO-A-99/03894.

The RAFT (reversible addition-fragmentation chain transfer) polymerization technique comprises blocking the growing free-radical species in the form of a bond of C—S type. Dithio compounds such as dithioesters (—C(S)S—), for example, dithiobenzoates, dithiocarbamates (—NC(S)S—) and dithiocarbonates (—OC(S)S—) (xanthates) are used to do this. These compounds make it possible to control the chain growth of a wide range of monomers. However, dithioesters inhibit the polymerization of vinyl esters, while dithiocarbamates are very weakly active towards methacrylates, which limits the application of these compounds to a certain extent. This technique is described, for example, in patent application WO-A-98/58974 from Rhodia and in the article “A more versatile route to block copolymers and other polymers of complex architecture by living radical polymerization: the RAFT process”, published in Macromolecules, 1999, Volume 32, pages 2071-2074. The already mentioned patent application WO-A-98/58974 and the patent application WO-A-99/31144 from CSIRO relate to the use of dithiocarbamates as “RAFT” reagents. By using these dithiocarbamates, various monomers are polymerized, including vinyl acetate.

The main advantages of the “RAFT” technique over the “ATRP” technique are that it does not require a metal catalyst and also that standard radical initiators may be used to initiate the reaction.

In the case of controlled free-radical polymerization, for example, of N-vinylpyrrolidone (NVP), the chain-transfer agents used in the case of “RAFT” polymerization may include, for example, diphenyldithiocarbamate derivatives.

By varying the ratio of the concentration of monomer to the concentration of chain-transfer agent, the molecular mass of the polymer may be modified.

The polymerization proceeds in several steps according to the general scheme:
a—in a first step, polymerization of the first monomer or mixture of monomers is performed to form a macroinitiator,
b—the polymers purified by precipitation are dried under vacuum,
c—next, in the second step, polymerization of the second block comprising a monomer or a mixture of monomers is performed at the end of the macroinitiator (formed in step a).

Steps b and c are repeated as many times as necessary according to the number of blocks.

An example of the process is the reverse ATRP polymerization process, which should not be confused with the standard ATRP process described above.

In this reverse ATRP process, the initiation is performed in a conventional manner with an initiator capable of giving radicals, for instance azobisisobutyronitrile (AIBN) or a peroxide, rather than using a specific initiator.

The presence of metal halides, such as CuBr₂ and of a ligand makes it possible to control the polymerization by reversibly scavenging the radicals formed.

The ligands include, for example, amine-based molecules, such as tris(dimethylaminoethyl)amine (Me₆TREN). Good control of the polymerization is observed, for example, with the AIBN/CuBr₂/Me₆TREN system.

Another example of the process is is the “RAFT” polymerization process.

Second Mode

The block polymers as disclosed herein may also be obtained by using the standard free-radical polymerization technique, by adding the monomers sequentially. In this case, only control of the nature of the blocks is possible (no control of the masses).

This involves polymerizing, in a first stage, a monomer M1 in a polymerization reactor; kinetically monitoring its consumption over time, and then, when M1 is approximately 95% consumed, introducing a new monomer M2 into the polymerization reactor.

A polymer of block structure of M1-M2 type is thus obtained.

More specifically, a first process for preparing a copolymer as described above comprising at least one block A and at least one block B and optionally at least one other block different from the blocks A and B, in which the polymerization is performed via the reverse atom transfer radical polymerization technique (reverse ATRP) comprises the following steps:
a) the at least one monomer from which the block A is prepared is polymerized in the presence of a chain-transfer agent, such as a transition metal halide, a free-radical initiator and a ligand, and in the presence or absence of a solvent, by means of which a macroinitiator or functional precursor capable of initiating a polymerization since it comprises the transfer agent function at its ends is obtained;
b) the at least one monomer from which the block B is prepared is polymerized at the end of the macroinitiator or precursor in the presence of a chain-transfer agent such as a transition metal halide, an initiator and a ligand, and in the presence or absence of a solvent, by means of which a diblock copolymer of structure A-b-B is obtained;
c) step b) is optionally repeated with the at least one monomer from which the block or the various other blocks of blocks A and B is (are) prepared, by means of which a triblock or multiblock copolymer is obtained.

It is possible to use difunctional initiators in order to obtain symmetrical triblock copolymers.

In one embodiment, the chain-transfer agents are chosen from halides of metals in the highest possible oxidation state, such as chosen from CuBr₂, CUCl₂, FeCl₂P-(phenyl)₃, FeCl₃ and RuCl₂P(phenyl)₃.

For example, the free-radical polymerization initiators are chosen from:
azo compounds, such as 2,2′-azobisisobutyronitrile (AIBN), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]-propionamide, 2,2′-azobis(2-methyl-N-hydroxyethyl)propionamide, 2,2′-azobis(N,N′-dimethyleneisobutyramidine) dichloride, 2,2′-azobis(2-amidinopropane) dichloride, 2,2′-azobis(N,N′-dimethyleneisobutyramide), 2,2′-azobis(2-methyl-N-[1,1-bis-(hydroxymethyl)-2-hydroxyethyl]propionamide), 2,2′-azobis(2-methyl-N-[1,1-bis(hydroxymethyl)ethyl]propionamide), 2,2′-azobis(2-methyl-N-(2-hydroxy-ethyl)propionamide), and 2,2′-azobis(isobutyramide) dihydrate;
hydrogen peroxides, such as tert-butyl hydroperoxide, cumene hydroperoxide, t-butyl peroxyacetate, t-butyl peroxybenzoate, t-butyl peroxyoctoate, t-butyl peroxyneodecanoate, t-butyl peroxyisobutarate, lauroyl peroxide, t-amyl peroxypivalate, t-butyl peroxypivalate, dicumyl peroxide, and benzoyl peroxide;
alkaline persulfates, such as potassium persulfate and ammonium persulfate;
redox systems comprising combinations such as:
   mixtures of hydrogen peroxide, alkyl peroxide, peresters, percarbonates and the like, and any iron salts, titanous salts, zinc formaldehyde sulfoxylate or sodium formaldehyde sulfoxylate, and reducing sugars;
   alkali metal or ammonium persulfates, perborate or perchlorate in combination with an alkali metal bisulfite, such as sodium metabisulfite, and reducing sugars, and
   alkali metal persulfates in combination with an arylphosphinic acid, such as benzenephosphonic acid and the like, and reducing sugars.

For example, the ligands are chosen from tetradentate ligands, such as 1,1,4,7,10,10-hemamethyltriethylenetetramine (HMTETA), 1,4,8,11-tetraazacyclo-tetradecane (cyclame), 1,4,8,11-tetramethyl-1,4,8,11-tetraazacyclotetradecane (Me₄ cyclame) and tris(dimethylaminoethyl)amine (Me₆TREN); hexadentate ligands, such as tetrakis(2-pyridyl)pyrazine (TPPY), N,N, N′,N′-tetrakis(2-pyridylmethyl)ethylenediamine (TPMEDA) and triphenylphosphine (TPP).

In one embodiment, the solvents are chosen from dioxane, tetrahydrofuran (THF), N-methylpyrrolidone and water, and mixtures thereof.

The “chain-transfer agents”, initiators, ligands and solvents may be identical or different in step a) and step b). In one embodiment, they are identical. For example, in the first and second steps a) and b), the chain-transfer agent is CuBr₂, the initiator is AIBN, the ligand is Me₆TREN and the solvent is dioxane.

This first process can make it possible to prepare any of the copolymers as disclosed herein, but it applies, for example, to the preparation of a copolymer in which block A is a copolymer derived from a major monomer that is N-vinylpyrrolidone and from a minor monomer that is chosen from acrylic acid, methacrylic acid, styrenesulfonic acid, 2-acrylamido-2-methylpropanesulfonic acid and tert-butyl acrylate, followed by hydrolysis, and block B is a homopolymer derived from a monomer chosen from methyl acrylate, t-butyl acrylate and methyl methacrylate.

A second process for preparing a copolymer as described above comprising at least one block A and at least one block B and optionally at least one other block (e.g., C, D) different from the blocks A and B, in which the polymerization is performed via the RAFT “Reversible Addition-Fragmentation Chain Transfer” polymerization technique includes the following two successive steps:
a) the at least one monomer from which block A is prepared is polymerized in the presence of a chain-transfer agent and an initiator, in a solvent or otherwise, by means of which a macroinitiator or precursor comprising the chain-transfer agent function at its ends is obtained;
b) the at least one monomer from which block B is prepared is polymerized at the end of the said macroinitiator or precursor, in the presence of an initiator, in a solvent or otherwise, by means of which a copolymer A-b-B is obtained;
c) step (b) is optionally repeated with the at least one monomer from which the other block(s) different from blocks A and B is (are) prepared, by means of which a triblock or multiblock copolymer is obtained.

In one embodiment, the chain-transfer agents are chosen from dithioesters (—C(S)S—) such as dithiobenzoates, dithiocarbamates (—NC(S)S—) and dithiocarbonates (—OC(S)S—) (xanthates).

For example, the chain-transfer agents are chosen from diethyl malonate diphenyl dithiocarbamate (DPCM), fluoroethyl acetate diphenyldithiocarbamate (DPFEM) and the xanthate of formula C₂H₅OC(S)SCH(CH₃)COOCH₃.

In one embodiment, the initiators are chosen from the compounds already mentioned above for the first process.

The solvents are chosen, for example, from dioxane, tetrahydrofuran, N-methylpyrrolidone and water, and mixtures thereof.

The initiators, chain-transfer agents and solvents may be identical or different in step a) and step b). In one embodiment, they are identical.

For example, in the first and the second step a) and b), the initiator is AIBN, the chain-transfer agent is chosen from diethyl malonate diphenyldithiocarbamate (DPCM), fluoroethyl acetate diphenyldithiocarbamate (DPFEM) and the xanthate of formula C₂H₅OC(S)SCH(CH₃)COOCH₃ according to the nature of the monomers to be polymerized, and the solvent is dioxane.

This second process can make it possible to prepare any one of the copolymers as disclosed herein, and it applies, for example, to the preparation of a copolymer in which block A is a copolymer derived from a major monomer chosen from N-vinylpyrrolidone and N-vinylcaprolactam, and from a minor monomer, which is chosen from acrylic acid, methacrylic acid, styrenesulfonic acid, 2-acrylamido-2-methylpropanesulfonic acid and tert-butyl acrylate, followed by hydrolysis, and block B is a homopolymer derived from a monomer chosen from methyl acrylate, t-butyl acrylate and methyl methacrylate.

Further disclosed herein are cosmetic or pharmaceutical compositions comprising the copolymer of specific structure as has been described above.

Generally, these compositions comprise from 0.1% to 60% by weight, such as from 0.5% to 50% by weight and further such as from 1% to 40% by weight of the copolymer as disclosed herein.

These compositions, which are, for example, cosmetic compositions, as disclosed herein further comprise a physiologically acceptable medium, i.e., a medium that is compatible with keratin materials, for instance the skin, the hair, the eyelashes, the eyebrows and the nails.

In general, it should be considered that the composition is physiologically acceptable.

The physiologically acceptable medium generally comprises at least one suitable physiologically acceptable solvent, in which the copolymer as disclosed herein is present in dissolved or dispersed form.

The composition, for example, the cosmetic composition, may thus comprise, as suitable solvents forming a hydrophilic phase, at least one solvent chosen from water and mixtures of water and of at least one hydrophilic organic solvent, chosen for instance from alcohols such as linear and branched lower monoalcohols comprising from 2 to 5 carbon atoms, for instance ethanol, isopropanol and n-propanol, and polyols, for instance glycerol, diglycerol, propylene glycol, sorbitol, pentylene glycol and polyethylene glycols. The hydrophilic phase may also comprise C₂ ethers.

The water or the mixture of water and of at least one hydrophilic organic solvent may be present in the composition as disclosed herein in an amount ranging from 0% to 90% (such as from 0.1% to 90%) by weight and such as from 0% to 60% by weight (such as from 0.1% to 60% by weight) relative to the total weight of the composition.

The physiologically acceptable medium of the composition may further comprise a fatty phase, comprising, for example, at least one component chosen from fatty substances that are liquid at room temperature (in general 25° C.) and fatty substances that are solid at room temperature, such as waxes, pasty fatty substances and gums, and mixtures thereof. These fatty substances may be of animal, plant, mineral or synthetic origin. This fatty phase may also comprise at least one lipophilic organic solvent.

As fatty substances that are liquid at room temperature, often known as oils, which may be used herein, mention may be made, for example, of: hydrocarbon-based oils of animal origin such as perhydrosqualene; hydrocarbon-based plant oils such as liquid triglycerides of fatty acids comprising from 4 to 10 carbon atoms, for instance heptanoic and octanoic acid triglycerides, or alternatively sunflower oil, corn oil, soybean oil, grape seed oil, sesame seed oil, apricot oil, macadamia oil, castor oil, avocado oil, caprylic/capric acid triglycerides, jojoba oil and shea butter oil; linear and branched hydrocarbons of mineral or synthetic origin such as liquid paraffins and derivatives thereof, petroleum jelly, polydecenes and hydrogenated polyisobutene such as parleam; synthetic esters and synthetic ethers, such as of fatty acids, for example, Purcellin oil, isopropyl myristate, 2-ethylhexyl palmitate, 2-octyldodecyl stearate, 2-octyidodecyl erucate and isostearyl isostearate; hydroxylated esters, for instance isostearyl lactate, octyl hydroxystearate, octyidodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters; fatty alcohols comprising from 12 to 26 carbon atoms, for instance octyidodecanol, 2-butyloctanol, 2-hexyldecanol, 2-undecylpentadecanol and oleyl alcohol; partially hydrocarbon-based and silicone-based fluoro oils; silicone oils, for instance linear or cyclic, volatile or non-volatile polymethylsiloxanes (PDMSs) that are liquid or pasty at room temperature, for instance cyclomethicones, dimethicones, optionally comprising a phenyl group, for instance phenyl trimethicones, phenyltrimethylsiloxydiphenyl siloxanes, diphenylmethyldimethyltrisiloxanes, diphenyl dimethicones, phenyl dimethicones and polymethylphenylsiloxanes; mixtures thereof.

These oils may be present in an amount ranging from 0.01% to 90% such as from 0.1% to 85% by weight, relative to the total weight of the composition.

The physiologically acceptable medium of the composition as disclosed herein may also comprise at least one organic solvent that is cosmetically and/or pharmaceutically acceptable (acceptable tolerability, toxicology and feel). The at least one organic solvent may generally be present in an amount ranging from 0% to 90% by weight, such as from 0.1% to 90% by weight and further such as from 10% to 90% by weight, and even further such as from 30% to 90% by weight, relative to the total weight of the composition.

As solvents that may be used in the composition as disclosed herein, mention may be made, for example, of acetic acid esters, for instance methyl acetate, ethyl acetate, butyl acetate, amyl acetate, 2-methoxyethyl acetate and isopropyl acetate; ketones, for instance methyl ethyl ketone and methyl isobutyl ketone; hydrocarbons, for instance toluene, xylene, hexane and heptane; aldehydes comprising from 5 to 10 carbon atoms; ethers comprising at least 3 carbon atoms; and mixtures thereof.

The waxes may be chosen from hydrocarbon-based waxes, fluoro waxes and silicone waxes and may be chosen from those of plant, mineral, animal and synthetic origins. In one embodiment, the waxes have a melting point of greater than 25° C. such as greater than 45° C.

As waxes that may be used in the composition as disclosed herein, mention may be made of beeswax, carnauba wax, candelilla wax, paraffin, microcrystalline waxes, ceresin and ozokerite; synthetic waxes, for instance polyethylene waxes and Fischer-Tropsch waxes, and silicone waxes, for instance alkyl dimethicones and alkoxy dimethicones comprising from 16 to 45 carbon atoms.

The gums are chosen generally, for example, from polydimethylsiloxanes (PDMSs) of high molecular weight and cellulose gums and polysaccharides and the pasty substances are chosen generally, for example, from hydrocarbon-based compounds, for instance lanolins and derivatives thereof, and PDMSs.

The nature and amount of the solid substances depend on the desired mechanical properties and textures. As a guide, the composition may comprise from 0 to 50% by weight such as from 1% to 30% by weight of waxes relative to the total weight of the composition.

The copolymer may be combined with at least one auxiliary film-forming agent.

The physiologically acceptable medium may thus also comprise at least one auxiliary film-forming agent. Such film-forming agent(s) may be chosen from any compound known to those skilled in the art as being capable of fulfilling the desired function, and may be chosen, for example, from plasticizers and coalescers.

The physiologically acceptable medium of the composition as disclosed herein may also comprise at least one dyestuff chosen from water-soluble dyes and pulverulent dyestuffs, for instance pigments, nacres and flakes that are well known to those skilled in the art. The at least one dyestuff may be present in the composition in an amount ranging from 0.01% to 50% by weight such as from 0.01% to 30% by weight, relative to the total weight of the composition.

The term “pigments” means white or colored, mineral or organic particles of any form, which are insoluble in the physiological medium and which are intended to color the composition.

The term “nacres” means iridescent particles of any form, produced, for example, in the shell of certain molluscs, or alternatively synthesized.

The pigments may be white or colored, and mineral and/or organic. Among the mineral pigments that may be mentioned, examples include titanium dioxide, optionally-surface-treated, zirconium oxide, cerium oxide, zinc oxide, iron oxide (black, yellow or red) and chromium oxide, manganese violet, ultramarine blue, chromium hydrate and ferric blue, and metal powders, for instance aluminium powder and copper powder.

Among the organic pigments that may be mentioned, examples include carbon black, pigments of D & C type, and lakes based on cochineal carmine or on barium, strontium, calcium or aluminium.

The nacreous pigments may be chosen from white nacreous pigments such as titanium-coated mica or bismuth oxychloride-coated mica, colored nacreous pigments such as titanium mica coated with iron oxides, titanium mica coated, for example, with ferric blue or with chromium oxide, titanium mica coated with an organic pigment of the abovementioned type and also nacreous pigments based on bismuth oxychloride.

The water-soluble dyes are chosen, for example, from beetroot juice and methylene blue.

The physiologically acceptable medium of the composition as disclosed herein may also comprise at least one filler, in an amount ranging, for example, from 0.01% to 50% by weight such as from 0.01% to 30% by weight, relative to the total weight of the composition. The term “fillers” means colorless or white, mineral or synthetic particles of any form, which are insoluble in the medium of the composition, irrespective of the temperature at which the composition is manufactured. These fillers serve, for example, to modify the rheology or the texture of the composition.

The fillers may be mineral or organic of any form, platelet, spherical or oblong, irrespective of the crystallographic form (for example lamellar, cubic, hexagonal, orthorhombic, etc.). Mention may be made, for example, of talc, mica, silica, kaolin, polyamide powder (Nylon®) (Orgasol® from Atochem), poly-α-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), polyorganosiloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids comprising from 8 to 22 carbon atoms such as from 12 to 18 carbon atoms, for example zinc stearate, magnesium stearate, lithium stearate, zinc laurate and magnesium myristate.

The physiologically acceptable medium of the composition as disclosed herein may also comprise at least one ingredient chosen from the ingredients commonly used in cosmetics or in pharmaceuticals, such as vitamins, thickeners, trace elements, softeners, sequestering agents, fragrances, acidifying and basifying agents, preserving agents, sunscreens, surfactants, antioxidants, agents for preventing hair loss, antidandruff agents, propellants, and film-forming and non-film-forming water-soluble or liposoluble polymers or polymers dispersed in water or a fatty phase, and mixtures thereof.

Needless to say, a person skilled in the art will take care to select this or these optional additional compound(s), and/or the amount thereof, such that the advantageous properties of the corresponding composition as disclosed herein are not, or are not substantially, adversely affected by the envisaged addition.

The composition, for example, the cosmetic composition, as disclosed herein may, for example, be in the form of a suspension, a dispersion, a solution, a gel, an emulsion, such as an oil-in-water (O/W) or water-in-oil (W/O) emulsion, or a multiple emulsion (W/O/W, polyol/O/W or O/W/0 emulsion), in the form of a cream, a paste, a mousse, a dispersion of vesicles, such as of ionic or nonionic lipids, a two-phase or multi-phase lotion, a spray, a powder, a paste, such as a soft paste (for example, a paste having a dynamic viscosity at 25° C. of about from 0.1 to 40 Pa·s at a shear rate of 200 s⁻¹ after measurement for 10 minutes in cone/plate geometry). The composition may be anhydrous, for example it may be an anhydrous paste.

A person skilled in the art may select the appropriate presentation form, and also the method for preparing it, on the basis of his general knowledge, taking into account firstly the nature of the constituents used, such as their solubility in the support, and secondly the intended use of the composition.

The composition as disclosed herein may be a makeup composition, for instance complexion products (foundations), rouges, eye shadows, lip products, concealer products, blushers, mascaras, eyeliners, eyebrow makeup products, lip pencils, eye pencils, nail products, such as nail varnishes, body makeup products or hair makeup products (hair lacquer or mascara).

The composition as disclosed herein may also be a hair product, such as for holding the hairstyle or for shaping the hair. The hair compositions are, for example, shampoos, hairsetting gels and lotions, blow-drying lotions, and fixing and styling compositions such as lacquers and sprays.

When the copolymer as disclosed herein is used in hair products, such as products for holding the hairstyle or for shaping the hair, it makes it possible to avoid “flaking” powdering.

The solutions may be packaged in various forms: for example, as gels or lotions, such as in vaporizers, in pump-dispenser bottles or in aerosol containers in order to allow the composition to be applied in vaporized form or in the form of a mousse. Such packaging forms are indicated, for example, when it is desired to obtain a spray or a mousse for fixing or treating the hair.

It has been seen hereinabove that the polymer as disclosed herein is, for example, suitable for use under such conditions since it is highly compatible with the at least one propellant gas used, for example, in aerosol containers. The at least one propellant may be chosen from dimethyl ether, C₃₋₅ alkanes, such as propane, n-butane and isobutane; 1,1-difluoroethane, mixtures of dimethyl ether and of C₃₋₅ alkanes, and mixtures of 1,1-difluoroethane and of dimethyl ether and/or of C₃₋₅ alkanes.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The present invention will now be described with reference to the following examples, which are given as non-limiting illustrations.

### EXAMPLES

In the examples below, polymers as disclosed herein are prepared via the reverse atom-transfer radical polymerization technique, also known as reverse ATRP, or via the RAFT (reverse addition-fragmentation chain transfer) polymerization technique.

In general, the formation of blocks in the case of the reverse ATRP process and of the RAFT process takes place in several steps.

Thus, in the case of a diblock copolymer, the process will be performed, for example, in the following manner:
a/the first step comprises a polymerization of the first monomer to form the macroinitiator or precursor;
b/the second step comprises the polymerization of the second monomer at the end of the macroinitiator, to form the diblock.

Between the two steps, a purification step may be necessary, for example, in the case of the reverse ATRP polymerization.

The general procedure followed in the examples will first be described, for each of the processes: process 1 (reverse ATRP polymerization) and process 2 (RAFT polymerization).

1. Process 1: Reverse ATRP

The reverse ATRP process is, for example, performed with tris(dimethylaminoethyl)amine (TREN Me₆) as ligand, using CuBr₂ and employing AIBN as initiator.

1.1 Starting Materials

The ethylenic monomer comprising a lactam ring, such as N-vinylpyrrolidone (VP), obtained from the company Aldrich®, is distilled under vacuum and stored under nitrogen at 0° C. before use.
The other monomers, such as tert-butyl acrylate, methyl methacrylate and methyl acrylate, obtained from the company Aldrich®, are dried over calcium hydride and distilled under vacuum.
The initiator azobisisobutyronitrile (AIBN) is recrystallized from methanol.
CuBr₂ (99.9%), CuBr and copper powder (99%) are obtained from the company Aldrich® and used as supplied.
The tris(dimethylaminoethyl)amine (TREN Me₆) is synthesized according to the procedures described in the literature, for example in the Matyjaszewski document ACS Symp. Ser. 2000; 760; 207.
All the solvents: THF, dioxane and DMF, are dried by distillation over CaH₂ before use.
   1.2. Polymerization

The general procedure is as follows:

1.2.1. First Step

Formation of the Macroinitiator or Precursor

CuBr2 and Me6 TREN are placed in a round-bottomed flask and dioxane is added thereto.

The solution is stirred for 30 minutes at 25° C.

The at least one monomer (according to the block prepared first and forming the macroinitiator) is added, followed by addition of the initiator AIBN.

The system is subjected to three cycles of vacuum-drying/argon. The solution is then heated in a thermostatically-regulated bath.

The viscosity increases. After reaction for the desired time, the system is cooled to room temperature.

The macroinitiator polymer is separated out by precipitation: it is diluted in chloroform and then precipitated from diethyl ether; this operation is repeated twice. The copper complex is separated from the polymer dissolved in the chloroform by passing through a neutral column. The polymer solution is then evaporated.

1.2.2. Second Step

Polymerization of the Second Block at the End of the Macroinitiator

The macroinitiator obtained in the first step is added to a solution of CuBr and Me₆TREN in dioxane under an inert atmosphere of nitrogen.

The required amount of the at least one monomer constituting the second block is added. The solution is heated. After the desired time, the system is cooled to room temperature.

The polymer is separated out by precipitation: it is diluted in chloroform and then precipitated from diethyl ether; this operation is repeated twice. The copper complex is separated from the polymer dissolved in the chloroform by passing through a neutral column. The polymer solution is evaporated.

The polymer is dried.

The isolated polymer is generally a white powder.

2. Process 2: RAFT

Ethylenic monomers comprising a lactam ring, for example vinyllactams, are polymerized with the following compounds as chain-transfer agents:

diethyl malonate diphenyldithiocarbamate (DPCM):

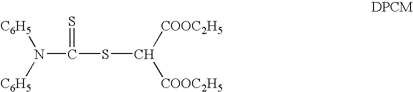

ethyl fluoroacetate diphenyldithiocarbamate (DPFEM)

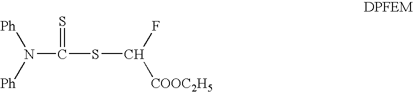

xanthate: C₂H₅O C(S)S C H(CH₃) COOCH₃

Starting Materials

The ethylenic monomer comprising a lactam ring, such as N-vinylpyrrolidone (VP) and N-vinylcaprolactam (VCap) is obtained from the company Aldrich® and is distilled under vacuum before use.
The other monomers, such as tert-butyl acrylate, methyl acrylate and methyl methacrylate, are obtained from the company Aldrich® and are dried over calcium hydride and distilled under vacuum.
The AIBN is obtained from the company Aldrich® and is recrystallized from methanol.
The diphenylamine, the CS₂ and the bromodiethyl malonate are obtained from the company Aldrich® and are used as received.
The solvents such as dioxane are dried by distillation over CaH₂ before use.
   2.1. Synthesis of the Chain-Transfer Agent
   2.1.1. Synthesis of Diethyl Malonate Diphenyldithiocarbamate (DPCM)

Diethyl malonate diphenyldithiocarbamate (DPCM) is synthesized from diphenylamine and bromodiethyl malonate in the following manner:

1.69 g of diphenylamine (10 mmol) in 10 ml of DMSO and 5 ml of THF are added at 0° C. to a solution of 0.625 g of NaH (purified by washing with dry hexane) in 5 ml of THF (dry). The reaction mixture is stirred for 1.5 hours to give a clear green solution. 1.2 eq of CS₂ (1.42 ml, 1.2 mmol) are added to this solution and the mixture is stirred for 30 minutes at 0° C. to obtain an orange-yellow solution of the sodium salt of diphenyl-dithiocarbamate.

Bromodiethyl malonate (10 mmol) is added to the above solution at −20° C. and the temperature of the reaction mixture is brought slowly up to room temperature. After stirring for 2 hours at room temperature, the reaction mixture is treated with water and extracted with ether. The ether layer is dried over MgSO₄ and concentrated.

Yield: 51%

The purity of the product was checked by NMR.

2.1.2. Synthesis of Fluoroethyl Acetate Diphenyldithiocarbamate (DPFEA)

Sodium hydride (7 mmol, 0.168 g, 1.3 eq in 5 ml of THF) is placed in a flame-dried flask and stirred at 0° C. Diphenylamine (5.4 mmol, 1 eq) in 5 ml of THF and 10 ml of DMSO is added dropwise to this mixture and stirred for 1 hour. Carbon disulfide (2.3 eq) is added to the solution at 0° C., and stirring is continued for a further half an hour. The temperature of the solution is reduced to 18° C. and one equivalent of fluoroethyl acetate is added. After addition, the reaction mixture is warmed slowly to room temperature and stirred for half an hour at room temperature. The product obtained is hydrolysed by adding water and the organic layer is extracted with ether. The ether extract is concentrated to give yellow crystals of (DPFEA) and the purity of the product is checked by NMR analysis.

2.2. Polymerization

2.2.1. The general procedure for the polymerization is the following, whether the chain-transfer agent is diethyl malonate diphenyldithiocarbamate or fluoroethyl acetate dithiocarbamate.

In the text hereinbelow, vinylpyrrolidone is mentioned, but the preparation process can be generalized to any ethylenic monomer comprising a lactam ring.

First Step

### Preparation of the Precursor (or Macroinitiator): Copoly(vinylpyrrolidone/minor monomer)

The polymerization of vinylpyrrolidone (VP) and of a minor monomer (for example tert-butyl acrylate) (block A) using diethyl malonate diphenyldithiocarbamate as RAFT reagent is performed using AIBN as initiator.

In a typical experiment, vinylpyrrolidone (VP), the diethyl malonate diphenyldithiocarbamate (chain-transfer agent/monomer ratio=1/100), the AIBN (10% of the chain-transfer agent) and dioxane (ratio of 1/1 by volume relative to the monomer) are placed in a Schlenk tube. The reaction mixture is degassed by three freeze-pump-thaw cycles and then sealed hermetically under vacuum and heated in a bath at a constant temperature of 80° C.

The “active” macroinitiator, which may be represented by ‘X PVP X with X=(phenyl)₂NC(S)S—, and X’=—CH(COOC₂H₅)₂, obtained is purified by repeated precipitations from ethyl ether and is dried under vacuum.

Second Step

Polymerization of the Second Block at the End of the Macroinitiator poly(vinylpyrrolidone/minor monomer)

The polymerization of the second monomer forming the block B (which is, for example, methyl methacrylate) takes place in the presence of the copolymer macroinitiator poly(vinylpyrrolidone/minor monomer) above, in 1.5 ml of dioxane and in the presence of AIBN (0.1 mol % relative to the total amount of monomer and of macroinitiator).

The reaction mixture is subjected to three freeze-pump-thaw cycles and heated in an oil bath at 80° C. with stirring for 20 hours.

Once the reaction is complete, the reaction mixture is dissolved in dichloromethane (minimum amount required for dissolution) and is precipitated from ether.

The cloudy ether solution is filtered and concentrated by flash evaporation, and is added to pentane; the precipitate obtained is dried under vacuum at about 70° C. for 8 hours.

2.2.2. As a variant, the polymerization may be performed in the following manner: the synthesis of a poly(methyl methacrylate) macroinitiator (block B) is first performed, for example, and the polymerization of block (A) mixture of vinylpyrrolidone and monomers: minor monomer such as tert-butyl acrylate is then performed at the end of this macroinitiator.

The same procedure as in paragraph 2.2.1. is followed.

2.2.3. Polymerization Using a Xanthate as Chain-Transfer Agent: Synthesis without an Intermediate Purification Step (“One-Pot Synthesis”)

N-Vinylpyrrolidone (4 ml, 3.74 eq; 10-2 mol) (the procedure may also be applied to another ethylenic monomer comprising a lactam ring), the minor monomer (e.g., tert-butyl acrylate), the chain-transfer agent (above xanthate, 0.0839 g, chain-transfer agent/monomer ratio=about 1/100 on a molar basis) and dioxane (4 ml) are placed in a predried round-bottomed flask and AIBN (0.0061 g, 10 mol % of the chain-transfer agent) is added thereto under nitrogen.

The reaction mixture is subjected to three freeze-pump-thaw cycles and is heated in an oil bath thermostatically maintained at 80° C., with stirring. The reaction is stopped after 14 hours by cooling in liquid nitrogen.

The second monomer (forming the block B), for example butyl acrylate (3 ml), and dioxane (3 ml) are added to this reaction mixture at room temperature under nitrogen. The reaction mixture is again subjected to three freeze-pump-thaw cycles and is heated at 80° C. for a further 24 hours.

Once the reaction is complete, the reaction mixture is dissolved in dichloromethane (minimum amount required for dissolution) and is precipitated from ether. The cloudy ether solution is filtered and concentrated by flash evaporation, it is added to pentane and the precipitate obtained is dried under vacuum at about 70° C. for 8 hours.

Characterizations

The conversion is measured by weighing the polymer.
The mass of the vinyllactam macroinitiator, for example, copolymer poly(vinylpyrrolidone/minor monomer), is determined by gas chromatography GC (Varian 9012® machine) with G4000 G3000 G2500 TSK® gel columns, equipped with a Varian® RI 4 infrared detector. The eluent is an 80/20 water/methanol mixture containing 0.1 M sodium nitrate. The flow rate of the eluent is 0.5 ml/minute. The calibration is performed with poly(ethylene oxide) standards.

The mass measurement is therefore only a comparative measurement and the values do not represent the actual molecular masses. However, the results may be used to note the trend in the change of the molecular masses and also to determine the mass dispersity of the chains.
The mass of the macroinitiator (methyl polymethacrylate, for example) is determined by GC (Varian® 9012) with G4000 G3000 G2500 TSK® gel columns, equipped with a Varian® RI 4 infrared detector. The eluent is THF. The standard is polystyrene.
The overall mass (GC) of the copolymer is determined by GC chromatography in solvent phase (Varian® 9012) with G4000 G3000 G2500 TSK® gel columns, equipped with a Varian® RI 4 infrared detector. The eluent is THF. The standard is polystyrene.
The proportion of the various blocks is determined by ¹H NMR (Bruker®, 200 MHz), by determining the ratio of the areas of the peaks corresponding to the monomers of the various blocks (i.e., on the one hand MMA and on the other hand VP/minor comonomer). The overall mass (NMR) of the copolymer is deduced with these values and the mass of the macroinitiator determined above.

After having described above the general procedure for the synthesis of the copolymers as disclosed herein, examples concerning the preparation of a specific copolymer as disclosed herein (Example 1) and also one comparative example are given below.

### Comparative Example

In this example, a copolymer (not in accordance with the invention) comprising a polyvinylpyrrolidone block and a poly(methyl methacrylate) block: (PVP) 80%-b-(PMMA) 20% was prepared according to process 1 (reverse ATRP) described above.

First Step

### Synthesis of the Polyvinylpyrrolidone-Br Macroinitiator: PVP—Br

CuBr₂ and Me₆TREN were placed in a 50 ml round-bottomed flask and 5 ml of dioxane were added.

Vinylpyrrolidone (VP) iwas then added, followed by addition of AIBN (azobisisobutyronitrile).

The dioxane/monomer ratio was 1/1 by volume.

The AIBN/CuBr₂/CuO/Me₆TREN proportions were: 1/1.5/0.15/3.

The reaction was performed at a temperature of 80° C. for 3 hours 45 minutes. The conversion reaches 84%.

Second Step

### Synthesis of the Copolymer: Polymerization of Methyl Methacrylate (MMA) at the End of the Polyvinylpyrrolidone Macroinitiator (PVP—Br)

The PVP macroinitiator (PVP—Br) was added to CuBr (ratio relative to 1/1.5 PVP—Br) and to Me₆TREN (1/3 relative to CuBr) in dioxane under a nitrogen atmosphere.

Polyvinylpyrrolidone macroinitiator PVP—Br: 0.026 mM.

Methyl methacrylate (MMA): 3.87 mM.

The reaction was performed at a temperature of 100° C. for 3 hours 45 minutes.

The conversion is 60%.

The characteristics of the macroinitiator and of the final copolymer are summarized in the following table:

| | | Mn (PVP | | | |
|---|---|---|---|---|---|
| | | macro- | % PVP/ | % PMMA/ | Mn PMMA |
| | VP/ | initiator) | total | total | deduced |
| Copolymer | AIBN | (GC) | weight | weight | by NMR |
| (PVP) | 1/0.25 | 38 700 | 80.2% | 19.8% | 9600 |
| 80%--b-- | | | | | |
| (PMMA) | | | | | |
| 20% | | | | | |

Vinylpyrrolidone = VP
Azobisisobutyronitrile = AIBN
Mₙ = number-average molecular mass

The polymer obtained is dissolved in water by adding water.

The polymer is water-insoluble; specifically, the solution is milky and unstable.

### Example 1

In this example, a copolymer (in accordance with the invention) comprising a block A (80% by weight) comprising a copolymer of 80% vinylpyrrolidone (VP) and 20% acrylic acid, and a block B (20% by weight) comprising poly(methyl methacrylate) was prepared. This poly(VP: 80%/acrylic acid: 20%) 80%-b-(PMMA) 20% polymer resulted from the hydrolysis of t-butyl acrylate of the following polymer: poly(VP: 70%/t-butyl acrylate: 30%) 80%-b-(PMMA) 20%.

1) Synthesis of the polymer poly(VP: 70%/t-butyl acrylate: 30%) 80%-b-(polymethyl methacrylate) 20%.

The desired polymer was prepared via a process similar to the one described in paragraph. 2.2.3. above. The monomer mixture was tailored for obtaining the desired polymer.

The N-vinylpyrrolidone (VP) and the tert-butyl acrylate (3.74×10⁻² mol in total), the chain-transfer agent (xanthate described above, 0.0839 g, chain-transfer agent/monomer ratio: about 1/100) and dioxane (4 ml) were placed in a predried flask, and AIBN (0.0061 g, 10 mol % relative to the chain-transfer agent) was added to the flask under nitrogen. This reaction mixture was subjected to three freeze-pump-thaw cycles and was heated in an oil bath thermostatically maintained at 80° C., with stirring.

The reaction was stopped after 14 hours by cooling in liquid nitrogen.

The monomer of the second block (methyl methacrylate) (3 ml) and dioxane (3 ml) were added to this reaction mixture at room temperature, under nitrogen. The reaction mixture was again subjected to three freeze-pump-thaw cycles and was heated at 80° C. for a further 24 hours.

Once the reaction was complete, the reaction mixture was dissolved in dichloromethane (minimum amount required for dissolution) and was precipitated from ether.

The cloudy ether solution was filtered and concentrated by flash evaporation, and wass added to pentane; the precipitate obtained was dried under vacuum at about 70° C. for 8 hours, and was characterized by GC and NMR.

The characteristics of the reagents and of the process are summarized below.

Chain-transfer agent: xanthate C₂H₅OC(S)SCH(CH₃)COOCH₃.

Molar ratio: xanthate/monomers: 1/100.

AlBN/xanthate chain-transfer agent ratio: 10 mol %.

Monomers/AIBN ratio: 0.1

Reaction temperature: 80° C.

Solvent: dioxane.

Vinylpyrrolidone (VP): 0.027 mol, i.e. 3 g.
tert-Butyl acrylate (tBuA): 0.0098 mol, i.e. 1.254 g.

The characteristics of the copolymer are summarized in the following table:

| | VP/tBuA | Mn [poly(VP-co-tBuA)] | Conversion | % PMMA/total | Mn PMMA deduced | % [poly(VP-co- |
|---|---|---|---|---|---|---|
| Copolymer | by weight | measured by GC | of MMA | weight | by NMR | tBuA)]/total weight |
| Poly(VP: 70%/t-butyl | 70/30 | 6500 | 90% | 25 | 2160 | 75 |
| acrylate: 30%) 80% | | | | | | |
| b-(polymethyl | | | | | | |
| methacrylate) 20% | | | | | | |

Mn = number-average molecular mass

2) Hydrolysis of poly(VP: 70%/t-butyl acrylate: 30%) 80%-b-(polymethyl methacrylate) 20%.

The polymer poly(VP: 70%/t-butyl acrylate: 30%) 80%-b-(polymethyl methacrylate) 20% was dissolved in dichloromethane to 10%. A solution of trifluoroacetic acid was added to this solution in a proportion of 5 molar equivalents relative to the t-butyl acrylate units.

To 10 g of polymer (i.e. 0.018 mol of tBuA) were added: 0.09 mol, about 11 g, of trifluoroacetic acid.

The reaction was carried out for 6 hours at room temperature.

The final polymer according to the invention, poly(VP: 80%/acrylic acid: 20%) 80%-b-(PMMA) 20%, was thus obtained.

The solubility of the polymer is assessed in the following test.

The polymer prepared above was neutralized by adding aqueous sodium hydroxide solution (1 M) to a proportion of 100% neutralization of the acrylic acid units.

The polymer according to the invention dissolves in water, unlike the polymer not in accordance with the invention, of the comparative example.

A film is made from the solution.

What is claimed is:

1. A linear block ethylenic copolymer comprising:
at least one block A that is obtained from monomers comprising
from 52% to 99% by weight of at least one ethylenic monomer comprising a lactam ring, chosen from monomers of formula (I) below: 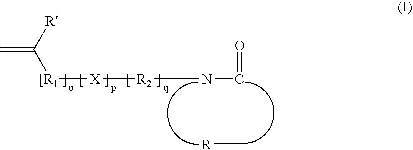
wherein:
R is a group —(CH₂)ₙ—, wherein at least one of the carbon atoms is optionally replaced with an atom chosen from nitrogen and oxygen atoms and is optionally substituted with at least one C₁ to C₆ alkyl group, and n is an integer from 3 to 12;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

2. The copolymer according to claim 1, wherein in formula (I), o is 0, p is 1, q is 1, R₂ is —CH₂CH₂—; X is chosen from —COO— and —CONH—; and R is chosen from —(CH₂)₃—, —(CH₂)₅—, and —CH₂CH₂NH—.

3. The copolymer according to claim 1, wherein the at least one ethylenic monomer comprising a lactam ring is chosen from vinyllactams of formula (II) below: 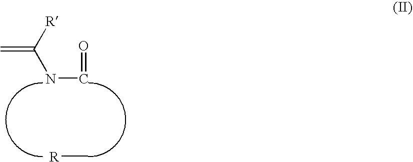
wherein:
R is a group —(CH₂)ₙ—, wherein at least one of the carbon atoms is optionally replaced with an atom chosen from nitrogen and oxygen atoms and is optionally substituted with at least one alkyl group chosen from C₁ to C₆ alkyl groups, and n is an integer from 3 to 12; and
R′ is chosen from H and a methyl group.

4. The copolymer-according to claim 1, wherein in formula (I), R is —CH₂—CH₂—NH— or —(CH₂)ₙ—, wherein n is an integer from 3 to 5.

5. The copolymer according to claim 3, wherein in formula (II), R is —CH₂—CH₂—NH— or —(CH₂)ₙ—, wherein n is an integer from 3 to 5.

6. The copolymer according to claim 1, wherein the copolymer is a film-forming copolymer.

7. The copolymer according to claim 1, wherein the copolymer has a number-average molecular mass ranging from 4000 to 1 000 000.

8. The copolymer according to claim 7, wherein the copolymer has a number-average molecular mass ranging from 4000 to 800 000.

9. The copolymer according to claim 8, wherein the copolymer has a number-average molecular mass ranging from 4000 to 500 000.

10. The copolymer according to claim 1, wherein the at least one block A is present in an amount of at least 50% by weight relative to the total weight of the copolymer.

11. The copolymer according to claim 10, wherein the at least one block A is present in an amount ranging from 50% to 99% by weight relative to the total weight of the copolymer.

12. The copolymer according to claim 11, wherein the at least one block A is present in an amount ranging from 55% to 95% by weight relative to the total weight of the copolymer.

13. The copolymer according to claim 12, wherein the at least one block A is present in an amount ranging from 60% to 90% by weight relative to the total weight of the copolymer.

14. The copolymer according to claim 1, wherein the at least one block B is present in an amount ranging from 1% to 50% by weight relative to the total weight of the copolymer.

15. The copolymer according to claim 14, wherein the at least one block B is present in an amount ranging from 5% to 45% by weight relative to the total weight of the copolymer.

16. The copolymer according to claim 15, wherein the at least one block B is present in an amount ranging from 10% to 40% by weight relative to the total weight of the copolymer.

17. The copolymer according to claim 1, wherein each of the at least one block A or the at least one block B has a number-average molecular mass ranging from 2000 to 1 000 000.

18. The copolymer according to claim 17, wherein each of the at least one block A or the at least one block B has a number-average molecular mass ranging from 2000 to 800 000.

19. The copolymer according to claim 18, wherein each of the at least one block A or the at least one block B has a number-average molecular mass ranging from 2000 to 500 000.

20. The copolymer according to claim 3, wherein the at least one ethylenic monomer comprising a lactam ring, chosen from those of formula (I) and formula (II), is present in the at least one block A in an amount ranging from 55% to 95% by weight relative to the total weight of the at least one block A.

21. The copolymer according to claim 20, wherein the at least one ethylenic monomer comprising a lactam ring, chosen from those of formula (I) and formula (II), is present in the at least one block A in an amount ranging from 60% to 80% by weight relative to the total weight of the at least one block A.

22. The copolymer according to claim 21, wherein the at least one ethylenic monomer comprising a lactam ring, chosen from those of formula (I) and formula (II), is present in the at least one block A in an amount ranging from 65% to 75% by weight relative to the total weight of the at least one block A.

23. The copolymer according to claim 22, wherein the at least one ethylenic monomer containing a lactam ring, chosen from those of formula (I) and formula (II) is present in the at least one block A in an amount of 70% by weight relative to the total weight of the at least one block A.

24. The copolymer according to claim 1, wherein the at least one block A has an overall glass transition temperature ranging from 0 to 250° C.

25. The copolymer according to claim 24, wherein the at least one block A has an overall glass transition temperature ranging from 0 to 220° C.

26. The copolymer according to claim 25, wherein the at least one block A has an overall glass transition temperature ranging from 5 to 200° C.

27. The copolymer according to claim 1, wherein the at least one block A is chosen from hydrophilic blocks.

28. The copolymer according to claim 1, wherein the at least one ethylenic monomer comprising a lactam ring of formula (I) is chosen from pyrrilidinoethyl acrylate and pyrrilidinoethyl methacrylate.

29. The copolymer according to claim 3, wherein the vinyllactams of formula (II) are chosen from N-vinylpyrrolidone (n=3), N-vinylpiperidinone (valerolactam) (n=4), N-vinylcaprolactam (n=5), N-vinylimidazolidinone wherein R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

30. The copolymer according to claim 1, wherein the at least one block A is chosen from random, alternating and gradient copolymers.

31. The copolymer according to claim 1, wherein the at least one ionic hydrophilic monomer is chosen from cationic monomers, anionic monomers, betaines and monomers that may be made ionic and hydrophilic following hydrolysis.

32. The copolymer according to claim 31, wherein the at least one ionic hydrophilic monomer is chosen from cationic monomers.

33. The copolymer according to claim 31, wherein the at least one ionic hydrophilic monomer is chosen from anionic monomers.

34. The copolymer according to claim 31, wherein the at least one ionic hydrophilic monomer is chosen from monomers that may be made hydrophilic following hydrolysis.

35. The copolymer according to claim 32, wherein the cationic monomers are chosen from 2-vinylpyridine; 4-vinylpyridine; dimethylaminoethyl (meth)acrylate; diethylaminoethyl (meth)acrylate; dimethylaminopropyl (meth)acrylamide; and salified and quaternized forms thereof.

36. The copolymer according to claim 35, wherein the salified forms are chosen from salts of mineral acids and salts of organic acids.

37. The copolymer according to claim 36, wherein the mineral acids are chosen from sulfuric acid and hydrochloric acid.

38. The copolymer according to claim 33, wherein the anionic monomers are chosen from acrylic acid, methacrylic acid, crotonic acid, maleic anhydride, itaconic acid, fumaric acid, maleic acid and vinylbenzoic acid; styrenesulfonic acid, 2-acrylamido-2-methylpropanesulfonic acid, and vinylphosphonic acid sulfopropyl methacrylate, and the salts thereof.

39. The copolymer according to claim 34, wherein the monomers that may be made hydrophilic following hydrolysis are chosen from monomers of (meth)acrylic ester that can be hydrolyzed to acids.

40. The copolymer according to claim 39, wherein the monomers that may be made hydrophilic following hydrolysis are chosen from ethyl, tert-butyl and benzyl (meth)acrylates.

41. The copolymer according to claim 1, wherein the at least one block B is obtained from at least one ethylenic monomer chosen from allylic monomers, acrylates, methacrylates, acrylamides, methacrylamides and vinyl monomers, and mixtures thereof, and optionally at least one ethylenic monomer comprising a lactam ring, of formula (I), wherein the optional at least one ethylenic monomers of formula (I) is present in the at least one block B in an amount of less than 50% by weight relative to the total weight of the at least one block B.

42. The copolymer according to claim 41, wherein the optional at least one ethylenic monomer of formula (I) is present in the at least one block B in an amount of less than or equal to 45% by weight relative to the total weight of the at least one block B.

43. The copolymer according to claim 42, wherein the optional at least one ethylenic monomer of formula (I) is present in the at least one block B in an amount of less than or equal to 40% by weight relative to the total weight of the at least one block B.

44. The copolymer according to claim 43, wherein the optional at least one ethylenic monomer of formula (I) is present in the at least one block B in an amount of less than or equal to 30% by weight relative to the total weight of the at least one block B.

45. The copolymer according to claim 1, wherein the monomers from which the at least one block B is obtained are chosen from the following monomers:
C₂-C₁₀ ethylenic hydrocarbons;
acrylates of formula CH₂═CHCOOR₃ and methacrylates of formula: 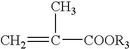
wherein R₃ is chosen from
linear and branched C₁-C₁₈ alkyl groups in which at least one hetero atom chosen from O, N, S and P is optionally intercalated, wherein at least one of the alkyl groups is also optionally substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group,
C₃ to C₁₂ cycloalkyl groups,
C₃ to C₂₀ aryl groups,
C₄ to C₃₀ aralkyl groups wherein the alkyl group is chosen from C₁ to C₈ alkyl groups,
4- to 12-membered heterocyclic groups comprising at least one hetero atom chosen from O, N and S, wherein the ring is aromatic or non-aromatic,
heterocyclylalkyl groups wherein the alkyl group is chosen from C₁-C₄ alkyl groups,
wherein at least one of the cycloalkyl, aryl, aralkyl, heterocyclic and heterocyclylalkyl groups is optionally substituted with at least one substituent chosen from hydroxyl groups, halogen atoms and linear and branched C₁-C₄ alkyl groups in which at least one hetero atom chosen from O, N, S and P is optionally intercalated, at least one of the alkyl groups also being optionally substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group,
(meth)acrylamides of formula: 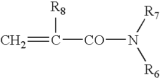
wherein:
R₈ is chosen from H and methyl; and
R₆ and R₇, which may be identical or different, are each chosen from:
a hydrogen atom;
linear and branched C₁-C₁₈ alkyl groups, in which at least one hetero atom chosen from O, N, S and P is optionally intercalated, at least one of the alkyl groups also optionally being substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group;
C₃ to C₁₂ cycloalkyl groups;
C₃ to C₂₀ aryl groups;
C₄ to C₃₀ aralkyl groups wherein the alkyl group is chosen from C₁ to C₈ alkyl groups;
4- to 12-membered heterocyclic groups comprising at least one hetero atom chosen from O, N and S, wherein the ring is aromatic or non-aromatic,
heterocyclylalkyl groups wherein the alkyl group is chosen from C₁-C₄ alkyl groups;
wherein at least one of the cycloalkyl, aryl, aralkyl, heterocyclic and heterocyclylalkyl groups is optionally substituted with at least one substituent chosen from hydroxyl groups, halogen atoms, and linear and branched C₁-C₄ alkyl groups in which at least one hetero atom chosen from O, N, S and P is optionally intercalated, at least one of the alkyl groups also being optionally substituted with at least one substituent chosen from hydroxyl groups, halogen atoms and groups Si(R₄R₅), wherein R₄ and R₅, which may be identical or different, are each chosen from C₁ to C₆ alkyl groups and a phenyl group;
the allylic compounds of formulae:
CH₂═CH—CH₂—R₉ and CH₂═C(CH₃)—CH₂—R₉,
and the vinyl compounds of formula:
CH₂═CH—R₈,
wherein R₉ is a group chosen from:
hydroxyl,
—Cl,
—NH₂,
—OR₁₀, wherein R₁₀ is chosen from a phenyl group and C₁ to C₁₂ alkyl groups, acetamide (—NHCOCH₃), and
—OCOR₁₁, wherein R₁₁ is chosen from:
linear and branched C₂-C₁₂ alkyl groups,
C₃ to C₁₂ cycloalkyl groups,
C₃ to C₂₀ aryl groups,
C₄ to C₃₀ aralkyl groups wherein the alkyl group is chosen from C₁ to C₈ alkyl groups,
4- to 12-membered heterocyclic groups comprising at least one hetero atom chosen from O, N and S, wherein the ring is aromatic or non-aromatic, and
heterocyclylalkyl groups wherein the alky group is chosen from C₁-C₄ alkyl groups,
wherein at least one of the cycloalkyl, aryl, aralkyl, heterocyclic and heterocyclylalkyl groups is optionally substituted with at least one substituent chosen from hydroxyl groups, halogen atoms, and linear and branched C₁-C₄alkyl groups in which at least one hetero atom chosen from O, N, S and P is optionally intercalated, at least one of the alkyl groups also being optionally substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group,
(meth)acrylic and (meth)acrylamide and vinyl monomers comprising at least one group chosen from fluoro and perfluoro groups; and
silicone (meth)acrylic and vinyl monomers.

46. The copolymer according to claim 45, wherein the C₂-C₁₀ ethylenic hydrocarbons are chosen from ethylene, isoprene and butadiene.

47. The copolymer according to claim 45, wherein the halogen atoms are chosen from Cl, Br, I and F.

48. The copolymer according to claim 45, wherein R₃ is chosen from methyl, ethyl, propyl, butyl, isobutyl, tert-butyl, ethylhexyl, octyl, lauryl and stearyl groups and from C₁₋₄ hydroxyalkyl groups and (C₁₋₄)alkoxy(C₁₋₄)alkyl groups.

49. The copolymer according to claim 48, wherein the C₁₋₄ hydroxyalkyl groups are chosen from 2-hydroxyethyl and 2-hydroxypropyl groups.

50. The copolymer according to claim 48, wherein the (C₁₋₄)alkoxy(C₁₋₄)alkyl groups are chosen from methoxyethyl, ethoxyethyl and methoxypropyl groups.

51. The copolymer according to claim 45, wherein, in defining R₃, R₆, R₇ and R₁₁, the C₃ to C₁₂ cycloalkyl groups are chosen from isobornyl and cyclohexyl groups.

52. The copolymer according to claim 45, wherein, in defining R₃, R₆, R₇ and R₁₁, the C₃ to C₂₀ aryl group is a phenyl group.

53. The copolymer according to claim 45, wherein, in defining R₃, R₆, R₇ and R₁₁, the C₄ to C₃₀ aralkyl groups are chosen from 2-phenylethyl and benzyl groups.

54. The copolymer according to claim 45, wherein, in defining R₃, R₆, R₇ and R₁₁, the heterocyclylalkyl groups are chosen from furfurylmethyl and tetrahydrofurfurylmethyl groups.

55. The copolymer according to claim 45, wherein R₃ is chosen from methyl, ethyl, propyl, isobutyl, n-butyl, tert-butyl, isobutyl, hexyl, ethylhexyl, octyl, lauryl, isooctyl, isodecyl, dodecyl, cyclohexyl, t-butylcyclohexyl, t-butylbenzyl, isobornyl, phenyl, furfurylmethyl, tetrahydrofurfurylmethyl 2-hydroxyethyl, 2-hydroxypropyl, 2-hydroxybutyl, methoxyethyl, ethoxyethyl, methoxyethyl, methoxypropyl and 2-ethylperfluorohexyl groups.

56. The copolymer according to claim 45, wherein R₃ for the acrylates is chosen from groups —(OC₂H₄)ₘ—OR″, wherein m is from 5 to 150 and R″ is chosen from H and C₁ to C₃₀ alkyl groups.

57. The copolymer according to claim 56, wherein R₃ for the acrylates is chosen from POE-methoxy and —POE-behenyl groups.

58. The copolymer according to claim 45, wherein R₆ and R₇ are each chosen from methyl, ethyl, n-butyl, t-butyl, isopropyl, isohexyl, isooctyl, isononyl and C₁₋₄ hydroxyalkyl groups.

59. The copolymer according to claim 45, wherein the (meth)acrylamides are chosen from (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide and N(2-hydroxypropyl methacrylamide).

60. The copolymer according to claim 45, wherein the allylic compounds are chosen from allylic esters and allyllic ethers.

61. The copolymer according to claim 45, wherein the vinyl compounds are chosen from vinyl esters, vinyl ethers, and vinyl monomers.

62. The copolymer according to claim 61, wherein the vinyl monomers are vinylcyclohexane and styrene.

63. The copolymer according to claim 61, wherein the vinyl esters are chosen from vinyl acetate, vinyl propionate, vinyl butyrate, vinyl ethylhexanoate₁, vinyl neononanoate and vinyl neododecanoate.

64. The copolymer according to claim 61, wherein the vinyl ethers are chosen from methyl vinyl ether, ethyl vinyl ether and isobutyl vinyl ether.

65. The copolymer according to claim 45, wherein the (meth)acrylic and (meth)acrylamide and vinyl monomers comprising at least one group chosen from fluoro and perfluoro groups are ethylperfluorooctyl methacrylate.

66. The copolymer according to claim 45, wherein the silicone (meth)acrylic and vinyl monomers are chosen from methacryloxypropyltris(trimethylsiloxy)silane, acryl-oxypropylpolydimethylsiloxane and silicone (meth)acrylamides.

67. The copolymer according to claim 1, wherein the monomers from which the at least one block B is obtained are chosen from monomers of which the glass transition temperature of the corresponding homopolymer is less than or equal to 110° C.

68. The copolymer according to claim 67, wherein the monomers from which the at least one block B is obtained are chosen from monomers for which the glass transition temperature of the corresponding homopolymer is less than or equal to 50° C.

69. The copolymer according to claim 68, wherein the monomers from which the at least one block B is obtained are chosen from monomers for which the glass transition temperature of the corresponding homopolymer is less than or equal to 20° C.

70. The copolymer according to claim 68, wherein the monomers for which the glass transition temperature of the corresponding homopolymer is less than or equal to 50° C. are chosen from methyl acrylate, ethyl acrylate, n-butyl acrylate, t-butyl acrylate, ethylhexyl acrylate, isobutyl acrylate, methoxyethyl acrylate, butyl methacrylate, ethoxyethyl methacrylate, POE methacrylate (n=8 to 10) and vinyl acetate.

71. The copolymer according to claim 45, wherein the monomers from which the at least one block B is obtained are chosen from methyl methacrylate, ethyl methacrylate, isobutyl methacrylate, furfuryl acrylate, isobornyl acrylate, tert-butylcyclohexyl acrylate, styrene and vinylcyclohexane.

72. The copolymer according to claim 1, wherein the monomers from which the at least one block B is obtained are chosen from hydrophilic monomers.

73. The copolymer according to claim 72, wherein the hydrophilic monomers are chosen from ionic monomers; nonionic monomers; and monomers that may be made hydrophilic following hydrolysis.

74. The copolymer according to claim 73, wherein the ionic monomers are chosen from cationic monomers, anionic monomers and betaines.

75. The copolymer according to claim 74, wherein the cationic monomers are chosen from 2-vinylpyridine; 4-vinylpyridine; dimethylaminoethyl (meth)acrylate; diethylaminoethyl (meth)acrylate; dimethylaminopropyl (meth)acrylamide; and salified and quaternized forms thereof.

76. The copolymer according to claim 74, wherein the anionic monomers are chosen from acrylic acid, methacrylic acid, crotonic acid, maleic anhydride, itaconic acid, fumaric acid, maleic acid and vinylbenzoic acid; styrenesulfonic acid, 2-acrylamido-2-methylpropanesulfonic acid, and vinylphosphonic acid sulfopropyl methacrylate, and the salts thereof.

77. The copolymer according to claim 74, wherein the betaines are chosen from ethylenic carboxybetaines and sulfobetaines.

78. The copolymer according to claim 77, wherein the ethylenic carboxybetaines and sulfobetaines are obtained by quaternization of ethylenically unsaturated monomers comprising at least one amine function with carboxylic acid sodium salts comprising a labile halogen, or with cyclic sulfones.

79. The copolymer according to claim 78, wherein the carboxylic acid sodium salt comprising a labile halogen is sodium chloroacetate.

80. The copolymer according to claim 78, wherein the cyclic sulfone is propane sulfone.

81. The copolymer according to claim 73, wherein the nonionic monomers are chosen from:
hydroxyalkyl (meth)acrylates, wherein the alkyl group comprises from 2 to 4 carbon atoms;
vinyllactams;
(meth)acrylamides and N—(C₁-C₄ alkyl)(meth)acrylamides; and
polysaccharide (meth)acrylates.

82. The copolymer according to claim 81, wherein the hydroxyalkyl (meth)acrylate is hydroxyethyl (meth)acrylate.

83. The copolymer according to claim 81, wherein the (meth)acrylamides and N—(C₁-C₄ alkyl)(meth)acrylamides are isobutylacrylamide.

84. The copolymer according to claim 81, wherein the polysaccharide (meth)acrylate is sucrose acrylate.

85. The copolymer according to claim 73, wherein the monomers that may be made hydrophilic following hydrolysis are chosen from monomers of (meth)acrylic ester that can be hydrolyzed to acids.

86. The copolymer according to claim 85, wherein the monomers that may be made hydrophilic following hydrolysis are chosen from ethyl, tert-butyl and benzyl (meth)acrylates.

87. The copolymer according to claim 1, wherein the copolymer is chosen from:
diblock copolymers (AB);
triblock copolymers (ABA, BAB, ABC and ACB), with C other than A or B;
multiblock copolymers comprising more than three blocks: (AB)ₙ, (ABA)ₙ, (BAB)ₙ, (ABC)ₙ and (ACB)ₙ, with C other than A or B, and multiblock copolymers comprising more than three different blocks: ABCD.

88. A cosmetic or pharmaceutical composition comprising at least one copolymer comprising:
at least one block A obtained from monomers comprising
from 52% to 99% by weight of at least one ethylenic monomer comprising a lactam ring, chosen from monomers of formula (I) below: 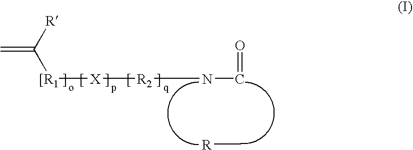
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

89. The composition according to claim 88, wherein the at least one copolymer is present in an amount ranging from 0.1% to 60% by weight relative to the total weight of the composition.

90. The composition according to claim 89, wherein the at least one copolymer is present in an amount ranging from 0.5% to 50% by weight relative to the total weight of the composition.

91. The composition according to claim 90, wherein the at least one copolymer is present in an amount ranging from 1% to 40% by weight relative to the total weight of the composition.

92. The composition according to claim 88, further comprising a physiologically acceptable medium in which the at least one copolymer is in a dissolved or dispersed form.

93. The composition according to claim 92, wherein the physiologically acceptable medium comprises at least one suitable solvent forming a hydrophilic phase, chosen from water and mixtures of water and of at least one hydrophilic organic solvent.

94. The composition according to claim 93, wherein the at least one hydrophilic organic solvent is chosen from alcohols.

95. The composition according to claim 94, wherein the alcohols are chosen from linear and branched lower monoalcohols comprising from 2 to 5 carbon atoms and polyols.

96. The composition according to claim 95, wherein the linear and branched lower monoalcohols comprising from 2 to 5 carbon atoms are chosen from ethanol, isopropanol and n-propanol.

97. The composition according to claim 95, wherein the polyols are chosen from glycerol, diglycerol, propylene glycol, sorbitol, pentylene glycol and polyethylene glycols.

98. The composition according to claim 93, wherein the hydrophilic phase further comprises C₂ ethers.

99. The composition according to claim 92, wherein the physiologically acceptable medium further comprises a fatty phase.

100. The composition according to claim 99, wherein the fatty phase comprises at least one fatty substance chosen from fatty substances that are liquid at room temperature, and fatty substances that are solid at room temperature, of animal, plant, mineral and synthetic origins.

101. The composition according to claim 92, wherein the physiologically acceptable medium further comprises at least one cosmetically and/or pharmaceutically acceptable organic solvent.

102. The composition according to claim 92, wherein the physiologically acceptable medium further comprises at least one auxiliary film-forming agent chosen from plasticizers and coalescers.

103. The composition according to claim 92, wherein the physiologically acceptable medium further comprises at least one dyestuff chosen from water-soluble dyes and pulverulent dyestuffs.

104. The composition according to claim 103, wherein the at least one dyestuff is chosen from pigments, nacres and flakes.

105. The composition according to claim 92, wherein the physiologically acceptable medium further comprises at least one filler.

106. The composition according to claim 92, wherein the physiologically acceptable medium further comprises at least one ingredient chosen from vitamins, thickeners, trace elements, softeners, sequestering agents, fragrances, acidifying and basifying agents, preserving agents, sunscreens, surfactants, antioxidants, agents for preventing hair loss, antidandruff agents, propellants, and film-forming or non-film-forming water-soluble or liposoluble polymers and polymers dispersed in water or a fatty phase, and mixtures thereof.

107. The composition according to claim 88, wherein the composition is in the form of a suspension, a dispersion, a solution, a gel, an emulsion, a cream, a mousse, a dispersion of vesicles, two-phase and multi-phase lotions, a spray, a powder, and a paste.

108. The composition according to claim 107, wherein the emulsion is chosen from oil-in-water (O/W) and water-in-oil (W/O) emulsions and multiple emulsions (W/O/W, polyol/O/W and O/W/O).

109. The composition according to claim 107, wherein the dispersion of vesicles is chosen from dispersions of at least one lipid chosen from ionic and non-ionic lipids.

110. The composition according to claim 107, wherein the paste is chosen from a soft paste and an anhydrous paste.

111. The composition according to claim 88, wherein the composition is a hair product.

112. The composition according to claim 111, wherein the hair product is chosen from a lacquer and a shampoo.

113. The composition according to claim 88, wherein the composition is a makeup composition.

114. The composition according to claim 113, wherein the makeup composition is a nail varnish.

115. The composition according to claim 88, wherein, in formula (I), o is 0, p is 1, q is 1, R₂ is —CH₂CH₂—; X is chosen from —COO— and —CONH— and R is chosen from —(CH₂)₃—, —(CH₂)₅—, and —CH₂CH₂NH—.

116. The composition according to claim 88, wherein the at least one ethylenic monomer comprising a lactam ring is chosen from vinyllactams of formula (II) below: 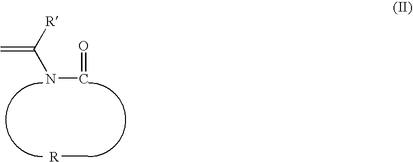
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups; and
R′ is chosen from H and a methyl group.

117. The composition according to claim 88, wherein in formula (I), R is chosen from —CH₂—CH₂—NH— and —(CH₂)ₙ—, wherein n is an integer from 3 to 5.

118. The composition according to claim 116, wherein in formula (II), R is chosen from —CH₂—CH₂—NH— and —(CH₂)ₙ—, wherein n is an integer from 3 to 5.

119. The composition according to claim 88, wherein the at least one copolymer is a film-forming copolymer.

120. The composition according to claim 88, wherein the at least one copolymer has a number-average molecular mass ranging from 4000 to 1 000 000.

121. The composition according to claim 88, wherein the at least one block A is present in an amount of at least 50% by weight relative to the total weight of the at least one copolymer.

122. The composition according to claim 88, wherein the at least one block B is present in an amount ranging from 1% to 50% by weight relative to the total weight of the at least one copolymer.

123. The composition according to claim 88, wherein each of the at least one block A or the at least one block B has a number-average molecular mass ranging from 2000 to 1 000 000.

124. The composition according to claim 116, wherein the at least one ethylenic monomer comprising a lactam ring, chosen from those of formula (I) and formula (II) is present in the at least one block A in an amount ranging from 55% to 95% by weight relative to the total weight of the at least one block A.

125. The composition according to claim 88, wherein the at least one block A has an overall glass transition temperature ranging from 0 to 250° C.

126. The composition according to claim 88, wherein the at least one block A is chosen from hydrophilic blocks.

127. The composition according to claim 88, wherein the at least one ethylenic monomer comprising a lactam ring of formula (I) is chosen from pyrrilidinoethyl acrylate and pyrrilidinoethyl methacrylate.

128. The composition according to claim 116, wherein the vinyllactams of formula (II) are chosen from N-vinylpyrrolidone (n=3), N-vinylpiperidinone (valerolactam) (n=4), N-vinylcaprolactam (n=5), N-vinylimidazolidinone wherein R is a —CH₂—CH₂—NH— group, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

129. The composition according to claim 88, wherein the at least one ionic hydrophilic monomer is chosen from cationic monomers, anionic monomers, betaines and monomers that may be made ionic and hydrophilic following hydrolysis.

130. The composition according to claim 88, wherein the at least one block B in the at least one copolymer is obtained from at least one ethylenic monomer chosen from allylic monomers, acrylates, methacrylates, acrylamides, methacrylamides and vinyl monomers, and mixtures thereof, and optionally at least one ethylenic monomer comprising a lactam ring, of formula (I), the optional at least one ethylenic monomers of formula (I) being present in the at least one block B in an amount of less than 50% by weight relative to the total weight of the at least one block B.

131. The composition according to claim 88, wherein the monomers from which the at least one block B is obtained are chosen from the following monomers:
C₂-C₁₀ ethylenic hydrocarbons;
acrylates of formula CH₂═CHCOOR₃ and methacrylates of formula: 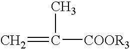
wherein R₃ is chosen from
linear and branched C₁-C₁₈ alkyl groups in which at least one hetero atom chosen from O, N, S and P is optionally intercalated, wherein at least one of the alkyl groups is also optionally substituted with at least one substituent chosen from hydroxyl groups, halogen atoms, and groups Si(R₄R₆), wherein R₄ and R₅, which may be identical or different, are each chosen from C₁ to C₆ alkyl groups and a phenyl group,
C₃ to C₁₂ cycloalkyl groups,
C₃ to C₂₀ aryl groups,
C₄ to C₃₀ aralkyl groups wherein the alkyl group is chosen from C₁ to C₈ alkyl groups,
4- to 12-membered heterocyclic groups comprising at least one hetero atom chosen from O, N and S, wherein the ring is aromatic or non-aromatic,
heterocyclylalkyl groups wherein the alkyl group is chosen from C₁-C₄ alkyl groups, wherein at least one of the cycloalkyl, aryl, aralkyl, heterocyclic and heterocyclylalkyl groups is optionally substituted with at least one substituent chosen from hydroxyl groups, halogen atoms and linear and branched C₁-C₄ alkyl groups in which at least one hetero atom chosen from O, N, S and P is optionally intercalated, at least one of the alkyl groups also being optionally substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group,
(meth)acrylamides of formula: 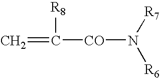
wherein:
R₈ is chosen from H and methyl; and
R₆ and R₇, which may be identical or different, are each chosen from:
a hydrogen atom;
linear and branched C₁-C₁₈ alkyl groups, in which at least one hetero atom chosen from O, N, S and P is optionally intercalated, at least one of the alkyl groups also optionally being substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group;
C₃ to C₁₂ cycloalkyl groups;
C₃ to C₂₀ aryl groups;
C₄ to C₃₀ aralkyl groups wherein the alkyl group is chosen from C₁ to C₈ alkyl groups;
4- to 12-membered heterocyclic groups comprising at least one hetero atom chosen from O, N and S, wherein the ring is aromatic or non-aromatic,
heterocyclylalkyl groups wherein the alkyl group is chosen from C₁-C₄ alkyl groups;
wherein at least one of the cycloalkyl, aryl, aralkyl, heterocyclic and heterocyclylalkyl groups is optionally substituted with at least one substituent chosen from hydroxyl groups, halogen atoms, and linear and branched C₁-C₄ alkyl groups in which at least one hetero atom chosen from O, N, S and P is optionally intercalated, at least one of the alkyl groups also being optionally substituted with at least one substituent chosen from hydroxyl groups, halogen atoms and groups Si(R₄R₅), wherein R₄ and R₅, which may be identical or different, are each chosen from C₁ to C₆ alkyl groups and a phenyl group;
the allylic compounds of formulae:
CH₂═CH—CH₂—R₉ and CH₂═C(CH₃)—CH₂—R₉,
and the vinyl compounds of formula:
CH₂═CH—R₉,
wherein R₉ is a group chosen from:
hydroxyl,
—Cl,
—NH₂,
—OR₁₀, wherein R₁₀ is chosen from a phenyl group and C₁ to C₁₂ alkyl groups,
acetamide (—NHCOCH₃), and
—OCOR₁₁, wherein R₁₁ is chosen from:
linear and branched C₂-C₁₂ alkyl groups,
C₃ to C₁₂ cycloalkyl groups,
C₃ to C₂₀ aryl groups,
C₄ to C₃₀ aralkyl groups wherein the alkyl group is chosen from C, to C₈ alkyl groups,
4- to 12-membered heterocyclic groups comprising at least one hetero atom chosen from O, N and S, wherein the ring is aromatic or non-aromatic, and
heterocyclylalkyl groups wherein the alky group is chosen from C₁-C₄ alkyl groups,
wherein at least one of the cycloalkyl, aryl, aralkyl, heterocyclic and heterocyclylalkyl groups is optionally substituted with at least one substituent chosen from hydroxyl groups, halogen atoms, and linear and branched C₁-C₄ alkyl groups in which at least one hetero atom chosen from O, N, S and P is optionally intercalated, at least one of the alkyl groups also being optionally substituted with at least one substituent chosen from hydroxyl groups, halogen atoms, and groups Si(R₄R₅), wherein R₄ and R₅, which may be identical or different, are each chosen from C₁ to C₆ alkyl groups and a phenyl group,
(meth)acrylic and (meth)acrylamide and vinyl monomers comprising at least one group chosen from fluoro and perfluoro groups; and
silicone (meth)acrylic and vinyl monomers.

132. The composition according to claim 88, wherein the monomers from which the at least one block B is obtained are chosen from monomers for which the glass transition temperature of the corresponding homopolymer is less than or equal to 110° C.

133. The composition according to claim 88, wherein the monomers from which the at least one block B is obtained are chosen from hydrophilic monomers.

134. The composition according to claim 133, wherein the hydrophilic monomers are chosen from ionic monomers; nonionic monomers; and monomers that may be made hydrophilic following hydrolysis.

135. The composition according to claim 88, wherein the at least one copolymer is chosen from:
diblock copolymers (AB);
triblock copolymers (ABA, BAB, ABC and ACB), with C other than A or B;
multiblock copolymers comprising more than three blocks: (AB)ₙ, (ABA)ₙ, (BAB)ₙ, (ABC)ₙ and (ACB)ₙ, with C other than A or B, and multiblock copolymers comprising more than three different blocks: ABCD.

136. A cosmetic process for making up or caring for a keratin material, comprising applying to the keratin material a composition comprising at least one copolymer comprising:
at least one block A that is obtained from monomers comprising
from 52% to 99% by weight of at least one ethylenic monomer comprising a lactam ring, chosen from monomers of formula (I) below: 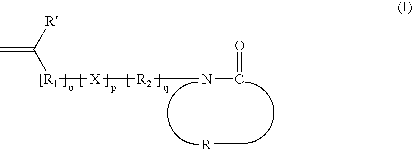
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

137. A method for improving the hairstyle hold of a hair lacquer, without tack, comprising including in the hair lacquer at least one copolymer comprising:
at least one block A that is obtained from monomers comprising
from 52% to 99% by weight of at least one ethylenic monomer comprising a lactam ring, chosen from monomers of formula (I) below: 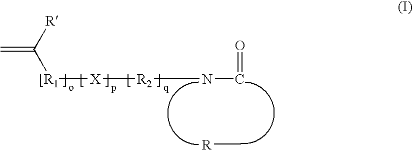
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

138. The method according to claim 137, wherein the method is for improving the hairstyle hold of a hair lacquer without having a tacky feel.

139. A method for improving the removal by shampooing of a hair composition, comprising including in the hair composition at least one copolymer comprising:
at least one block A that is obtained from monomers comprising
from 52% to 99% by weight of at least one ethylenic monomer comprising a lactam ring, chosen from monomers of formula (I) below: 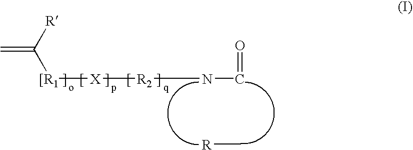
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

140. The method according to claim 139, wherein the hair composition is a hair lacquer.

141. A method for increasing the adhesion and the wear resistance of a nail varnish, without tack, comprising including in the nail varnish at least one copolymer comprising:
at least one block A that is obtained from monomers comprising
from 52% to 99% by weight of at least one ethylenic monomer comprising a lactam ring, chosen from monomers of formula (I) below: 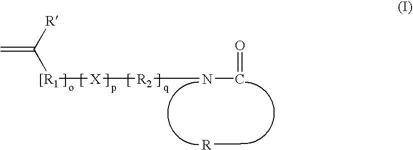
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

142. The method according to claim 141, wherein the method is for increasing the adhesion and the wear resistance of a nail varnish, without having a tacky feel.

143. A method for optimizing the adhesion to skin and the comfort of a makeup composition, comprising including in the makeup composition at least one copolymer comprising:
at least one block A that is obtained from monomers comprising
from 52% to 99% by weight of at least one ethylenic monomer comprising a lactam ring, chosen from monomers of formula (I) below: 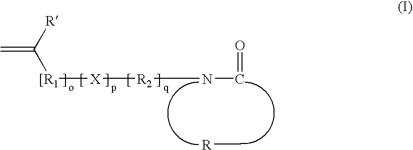
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

144. A method for reducing the tack of a cosmetic composition, comprising including in the cosmetic composition at least one copolymer comprising:
at least one block A that is obtained from monomers comprising
from 52% to 99% by weight of at least one ethylenic monomer comprising a lactam ring, chosen from monomers of formula (I) below: 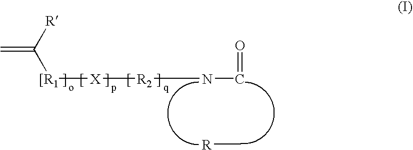
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

145. The method according to claim 144, wherein the method is for reducing the tacky feel of a cosmetic composition.

146. A method for reducing the tack of a cosmetic composition under conditions of high humidity, comprising including in the cosmetic composition at least one copolymer comprising:
at least one block A that is obtained from monomers comprising
from 52% to 99% by weight of at least one ethylenic monomer comprising a lactam ring, chosen from monomers of formula (I) below: 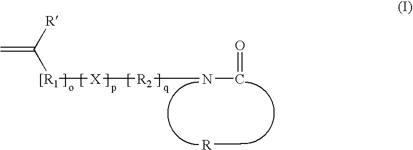
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO—and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof.

147. The method according to claim 146, wherein the method is for reducing the tacky feel of a cosmetic composition under conditions of high humidity.

148. The method according to claim 147, wherein conditions of high humidity are defined as a relative humidity (RH) value ranging from 50% to 100%.

149. A method for making a cosmetic composition for masking wrinkles, comprising including in the cosmetic composition at least one copolymer comprising:
at least one block A that is obtained from monomers comprising
from 52% to 99% by weight of at least one ethylenic monomer comprising a lactam ring, chosen from monomers of formula (I) below: 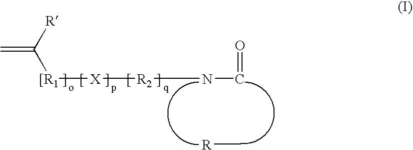
wherein:
R is a group —(CH₂)ₙ—, wherein n is an integer from 3 to 12, and wherein at least one of the carbon atoms is optionally replaced with an atom chosen from nitrogen and oxygen atoms and is optionally substituted with at least one alkyl group chosen from C₁ to C₆ alkyl groups;
R′ is chosen from H and a methyl group;
R₁ and R₂, which may be identical or different, are each chosen from linear, branched and cyclic C₁ to C₂₂ alkylene and aralkylene groups, wherein at least one of the carbon atoms is optionally replaced with an atom chosen from oxygen and nitrogen atoms;
X is chosen from —OCO—, —NHCO—, —COO— and —O—; and
o, p and q, which may be identical or different, are each chosen from 0 and 1; and
from 1% to 48% by weight of at least one ionic hydrophilic monomer;
and at least one block B that is obtained from monomers not comprising an ethylenic monomer comprising a lactam ring of formula (I), or comprising a minor proportion thereof, in an effective amount to give skin a smoothened appearance, without tautness.

150. The method according to claim 149, wherein the cosmetic composition is a make-up composition.
